(19) United States
(12) Patent Application Publication
(10) Pub. No.: US 2025/0250240 A1
(43) Pub. Date: Aug. 7, 2025

(54) PHENAZINE COMPOUND FOR MEASURING BACTERIAL EFFLUX

(71) Applicants: UNIVERSITE D'AIX-MARSEILLE, MARSEILLE (FR); INSTITUT NATIONAL DE LA SANTE ET DE LA RECHERCHE MEDICALE, Paris (FR); CENTRE NATIONAL DE LA RECHERCHE SCIENTIFIQUE, Paris (FR); ETAT FRANCAIS (MINIATÉRE DES ARMÉES) REPRÉSENTÉ PAR LA DIRECTION CENTRALE DU SERVICE DE SANTÉ DES AR, Paris cedex 15 (FR)

(72) Inventors: Frédéric BRUNEL, Arles (FR); Oliver SIRI, Belcodene (FR); Serhii KRYKUN, Rouen (FR); Mrunal PATIL, Marseille (FR); Michel CAMPLO, L'isle sur la sorgue (FR); Jean-Michel BOLLA, Marseille (FR); Jean-Manuel RAIMUNDO, Marseille (FR); Frédéric GARZINO, Marseille (FR); Eric GARNOTEL, Allauch (FR)

(73) Assignees: UNIVERSITE D'AIX-MARSEILLE, MARSEILLE (FR); INSTITUT NATIONAL DE LA SANTE ET DE LA RECHERCHE MEDICALE, Paris (FR); CENTRE NATIONAL DE LA RECHERCHE SCIENTIFIQUE, Paris (FR); ETAT FRANCAIS (MINIATÉRE DES ARMÉES) REPRÉSENTÉ PAR LA DIRECTION CENTRALE DU SERVICE DE SANTÉ DES AR, Paris cedex 15 (FR)

(21) Appl. No.: 18/730,523

(22) PCT Filed: Jan. 23, 2023
(86) PCT No.: PCT/EP2023/051578

§ 371 Date: Feb. 11, 2025

(30) Foreign Application Priority Data
Jan. 21, 2022 (FR) 2200522

(51) Int. Cl. C07D 241/46; C12Q 1/18; G01N 33/533

(52) U.S. Cl. CPC C07D 241/46; C12Q 1/18; G01N 33/533

(57) ABSTRACT

The invention relates to a compound of the following formula (I) in which X is an anion, in which R1 and R2 are either together —CH₃ or a C₁-C₄ alkyl such that, if R1 is a Cₙ₊ᵢ alkyl, R2 is a Cₙ alkyl, and vice versa, n being an integer ranging from 1 to 3, i being an integer ranging from 1 to 3 and in which n+i ranges from 1 to 4; and in which R3 is a —CH₃ or a C₂-C₈ alkyl, said C₂-C₈ alkyl being optionally substituted with one or more heteroatoms.

The invention concerns cyclic compounds, and in particular their use in measuring bacterial efflux.

In biology, efflux is a mechanism whereby cells release compounds that are toxic to their metabolism, such as antibiotics, heavy metals, drugs, etc., into the external environment. Efflux is an active, energy-dependent transport mechanism mediated by transmembrane proteins known as efflux pumps.

Many efflux systems have been described in prokaryotes, especially bacteria, but these mechanisms can also be observed in eukaryotes.

Due to the expulsion of compounds into the external environment, the mechanisms responsible for efflux contribute to the emergence of resistance to various pharmacological treatments, such as antibiotic resistance or resistance to cancer chemotherapy. The broad spectrum of certain efflux pumps observed in certain bacteria can lead to the emergence of multi-resistant phenotypes, making the treatment of infections extremely difficult.

There is therefore a need to measure efflux in order to anticipate bacterial resistance.

One aim of the invention is to provide compounds and methods for measuring bacterial efflux.

Another aim of the invention is to provide a method for assessing antibiotic resistance in bacteria.

The invention relates to a compound of the following formula I:

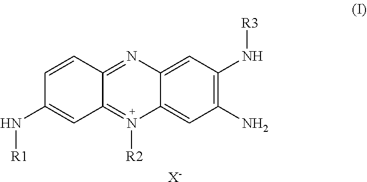

where X⁻ is a halogen atom, or an acceptable counter ion, in particular Cl,
where R1 and R2 are
   either together —CH₃, or —C₄H₉
   or a C1-C4 alkyl, such that if R1 is a Cn+i alkyl, R2 is a Cₙ alkyl, and vice versa, n being an integer ranging from 1 to 3, i being an integer ranging from 1 to 3 and where n+i ranges from 1 to 4;
   or R1 is —C₄H₇ and R2 is C₈H₁₇, and
where R3 is a —CH₃, or a C2-C8 alkyl, said C2-C8 alkyl being optionally substituted with one or more heteroatoms;
or a salt or solvate of this compound of formula I, or a protonated form thereof.

In other words, the invention relates to a compound of the above-mentioned formula I
where X— is a halogen atom, or an acceptable counter ion, in particular Cl,
where R1 and R2 are
   either together —CH₃, or —C₄H₉,
   or a C1-C4 alkyl, such that
   if R1 is a Cₙ₊ᵢ alkyl, R2 is a Cₙ alkyl,
   if R1 is a Cₙ alkyl, R2 is a Cₙ₊ᵢ alkyl,
n being an integer ranging from 1 to 3, i being an integer ranging from 1 to 3 and in which n+i ranges from 2 to 4;
or R1 is C₄H₉ and R2 is C₈H₁₇, and
where R3 is a —CH3, or a C2-C8 alkyl, said C2-C8 alkyl being optionally substituted with one or more heteroatoms;
or a salt or solvate of this compound of formula I, or a protonated form thereof.

Advantageously, the invention relates to a compound of the following formula I:

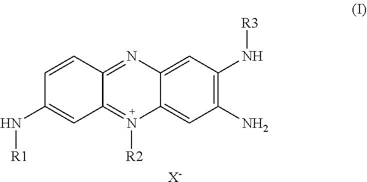

where X⁻ is a halogen atom, or an acceptable counter ion, in particular Cl,
where R1 and R2 are
   either together —CH₃,
   or a C1-C4 alkyl, such that if R1 is a Cₙ₊ᵢ alkyl, R2 is a Cₙ alkyl, and vice versa, n being an integer ranging from 1 to 3, i being an integer ranging from 1 to 3 and where n+i ranges from 1 to 4; and
   where R3 is a —CH₃, or a C2-C8 alkyl, said C2-C8 alkyl being optionally substituted with one or more heteroatoms;
   or a salt or solvate of this compound of formula I, or a protonated form thereof.

The invention is based on the inventors' surprising observation that the above-mentioned compounds of formula I are colored, fluorescent compounds that are efficiently effluxed by bacteria through the various efflux pumps, so that said compounds enable the bacterial efflux to be measured and the potential resistance of said bacteria to be measured.

Formula I compounds are phenaziniums, that is, cationic protonated forms of phenazines. Alkylated forms of the phenaziniums of the invention are also envisaged.

In compounds of formula I, X⁻ is an acceptable and in particular pharmaceutically acceptable counter-anion, that is, a counter-anion whose use is compatible with animal or bacterial physiology. Advantageous counter-anions are in particular counter-anions where X is a halogen atom such as F, Cl, Br or I, the advantageous halogen being Cl. Other possible counterions include chlorates, bromates, hydrogen carbonates, acetates, butyrates, formates, tartrates, aluminates, salicylates, mesylates, boron anions, phosphorus anions such as hexafluorophosphate (PF₆⁻) or tetrafluoroborate (BF₄⁻).

The compounds according to formula I are therefore phenaziniums where the groups R1, R2 and R3 are defined as follows:
either R1 and R2 are —CH₃ or —C₄H₉ groups,
or R1 and R2 are C1-C4 alkyls, that is, C1, C2, C3 or C4 alkyls,
preferably linear, so that R1 and R2 always have a different carbon atom. This is defined by the phase specifying that if R1 is a Cn alkyl, R2 is a Cn+i alkyl, and if R1 is a Cn+i alkyl, R2 is a Cn alkyl, where i is an integer ranging from 1 to 3 and n+i ranges from 2 to 4,
or R1 is —C₄H₇ and R2 is C₈H₁₇.

In other words, R1 and R2 are
together a —CH₃, or,
when R1 and R2 are linear C1-C4 alkyls,
R1=—CH₃ and R2=—C₂H₅ or R1=—C₂H₅ and R2=—CH₃, or
R1=—C₂H₅ and R2=—C₃H₇ or R1=—C₃H₇ and R2=—C₂H₅, or
R1=—C₃H₇ and R2=—C₄H₉ or R1=—C₄H₉ and R2=—C₃H₇.

Of course, when R1 or R3 are C3 alkyls, they can be C₃H₇ (that is, —CH₂—CH₂—CH₃) or CH(CH₃)₂, and when they are C4 alkyls they can be —C₄H₉ (that is, —CH₂—CH₂—CH₂—CH₃), —CH(CH₃)—CH₂—CH₃ or —CH₂—CH(CH₃)₂ or —C(CH₃)₃.

With regard to R3, this is a methyl (—CH₃) or C2-C8 alkyl, that is, an alkyl that is:
C1, that is —CH₃, or
C2, that is —C₂H₅ (that is —CH₂—CH₃), or
C3, that is —C₃H₇ (that is —CH₂—CH₂—CH₃) or —CH(CH₃)₂, or
C4, that is —C₄H₉ (that is —CH₂—CH₂—CH₂—CH₃), —CH(CH₃)—CH₂—CH₃ or —CH₂—CH(CH₃)₂ or —C(CH₃) 3, or
C5, that is —C5H11 (that is —CH₂—CH₂—CH₂—CH₂—CH₃), —CH(CH₃)—CH₂—CH₂—CH₃, —CH₂—CH(CH₃)—CH₂—CH₃, —CH₂—CH₂—CH(CH₃)₂, —C(CH₃)₂—CH₂—CH₃, —CH(CH₃)—CH(CH₃)—CH₃, —CH₂—C(CH₃) 3 or —CH(CH₃)—CH(CH₃)₂, or
C6, that is —C6H13 (that is —CH₂—CH₂—CH₂—CH₂—CH₂—CH₃), or a branched form, or
C7, that is —C7H15 (that is —CH₂—CH₂—CH₂—CH₂—CH₂—CH₂—CH₃), or a branched form, or
C8, that is —C₈H₁₇ (that is —CH2-CH2-CH2CH2-CH2-CH2-CH2-CH3), or a branched form.

When R3 is C2-C8 alkyl, each of the carbon atoms can be substituted by a heteroatom, that is an oxygen (O), nitrogen (N) or sulfur(S) atom, or even phosphorus (P). For example, a C4 alkyl may have one of its carbons substituted by an oxygen, giving the following R3 residues:
—O—CH₂—CH₂—CH₃, or
—CH₂—O—CH₂—CH₃, or
—CH₂—CH₂—O—CH₃, or
—CH₂—CH₂—NH—CH₃, or
—CH₂—CH₂—CH₂—OH.

These examples are illustrative and cannot limit the scope of the invention.

Protonated forms of the above-mentioned compound of formula I are also covered by the present invention.

Advantageously, the invention relates to the compound as defined above, where R1 and R2 are each a C2-C4 alkyl, such that:
if R1 is a Cn+i alkyl, R2 is a Cₙ alkyl, or
if R2 is a Cn+i alkyl, R1 is a Cₙ alkyl,
n ranging from 1 to 3, i being an integer ranging from 1 to 3 and n+i ranging from 1 to 4, and R3 is as defined below.

Advantageously, the invention relates to the compound as defined above, where R3 is a C2-C4 alkoxy group, in particular methoxyethyl, dimethylaminoethyl or methylaminexethyl.

Even more advantageously, the invention relates to the above-mentioned compound, said compound being selected from the group of compounds of formula I, wherein R1, R2 and R3 are together as indicated in each row of the following table:

TABLE 1

| compound | R1 | R2 | R3 |
|---|---|---|---|
| | methyl | methyl | propyl |
| | methyl | ethyl | propyl |
| | ethyl | methyl | propyl |
| | ethyl | propyl | propyl |
| | propyl | ethyl | propyl |
| | propyl | butyl | propyl |
| | butyl | propyl | propyl |
| | methyl | methyl | butyl |
| | methyl | ethyl | butyl |
| | ethyl | methyl | butyl |
| | ethyl | propyl | butyl |
| | propyl | ethyl | butyl |
| | propyl | butyl | butyl |
| #47 | butyl | propyl | butyl |
| | methyl | methyl | methoxyethyl |
| | methyl | ethyl | methoxyethyl |
| | ethyl | methyl | methoxyethyl |
| | ethyl | propyl | methoxyethyl |
| | propyl | ethyl | methoxyethyl |
| | propyl | butyl | methoxyethyl |
| #53 | butyl | propyl | methoxyethyl |
| #52 | butyl | propyl | octyl |
| #392 or #44 | butyl | octyl | methoxyethyl |
| #29 | butyl | butyl | propyl |

Note that compound #392 is also named #44

Even more advantageously, the invention relates to the above-mentioned compound, said compound being selected from compounds of the following formulae 47, 52, 53, 392 and 29:

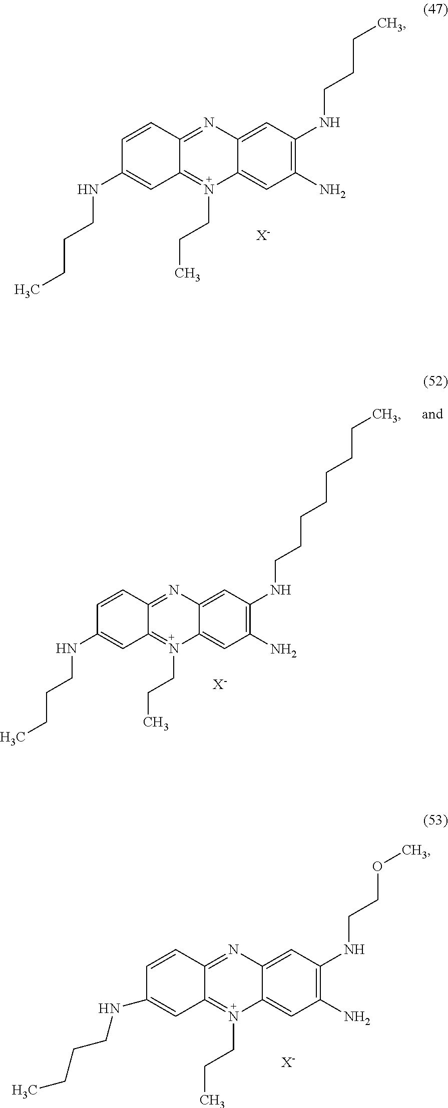

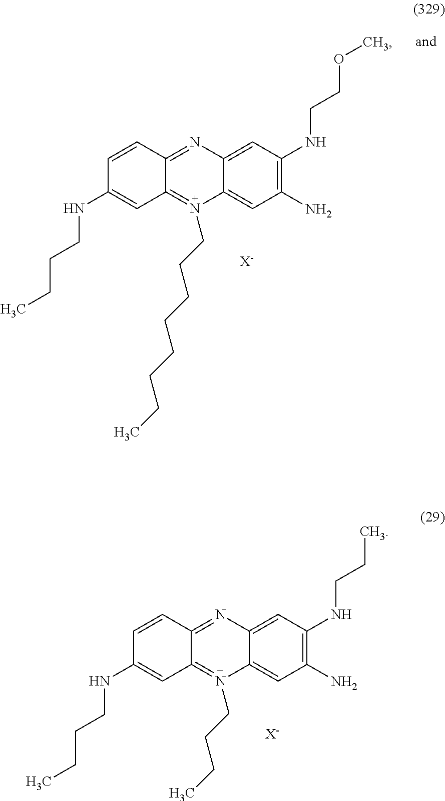

These three compounds are the most advantageous of the invention and their synthesis is described in detail in Example 1, below.

Advantageously, the invention relates to the compound as defined above, as a medicament, or for its use as a medicament. It is also envisaged to use the compound as defined above, for the manufacture of a medicament.

Also envisaged is a pharmaceutical composition comprising as active substance a compound as defined above, in association with a pharmaceutically acceptable vehicle. Preferably, the compound of formula (I) as defined above, or its pharmaceutically acceptable salt, is administered or administrable in a unit dose, or is packaged in a unit dose, of from about 50 mg to about 1500 mg, particularly from about 150 to about 200 mg. Also preferably, the compound of formula (I) as defined above, or its pharmaceutically acceptable salt, is administered or administrable with a dosage regimen of from 50 mg/d to about 1500 mg/d, particularly from about 150 mg/d to about 200 mg/d. Preferably, the compound of formula (I) as defined above, or its pharmaceutical salt, is administered or administrable in a form suitable for oral or injectable administration. More preferably, the compound of formula (I) as defined above, or its pharmaceutically acceptable salt, is administered or administrable in the form of a powder, tablets, gelatin capsules or sachets.

Even more advantageously, the aforementioned compound or pharmaceutical composition is used in the treatment, or for the treatment, of Gram-positive bacterial infections. The aforementioned compound has antibacterial properties that may prove useful in the treatment of infections.

More advantageously, the aforementioned compound is used as part of antibacterial therapies, to treat pathologies or infections linked to contamination by Gram-positive bacteria that are not capable of effluxing compounds, or bacteria that express few or no pumps responsible for antibiotic resistance.

In another aspect, the aforementioned compound, which possesses antibiotic, or at least antibacterial, properties, can be used as part of the decontamination of inert surfaces infected, or contaminated, with bacteria. The compound is then brought into contact with the contaminated surface to kill and/or inhibit the growth of the contaminating bacteria. The compound can be used in the form of a powder to be spread over the surface to be decontaminated, or in the form of a solution that can be poured or sprayed onto the surface to be decontaminated.

It is also envisaged to use the above-mentioned compound, in order to stain or to carry out a stained or fluorescent marking, of Gram-positive bacteria, and more particularly Gram-positive bacteria which are not capable of effluxing compounds, that is, bacteria which express few or no pumps responsible for multiple drug resistance (efflux pumps).

The invention also concerns the use of a compound as defined above, to evaluate or measure, that is to qualitatively or quantify observe the efflux of said compound by a bacterium.

As shown in the examples, the compound according to the invention is capable of being expelled from bacteria by the efflux mechanism. Also, since the compound according to the invention is stained and/or fluorescent, it is easy to follow its efflux from inside the bacteria to the outside.

It is also possible to use the compound of the invention to identify whether or not a bacterial isolate is overproducing its efflux pumps. Also, by measuring or evaluating the efflux of the compound of the invention, it is possible to determine whether a pump is overproduced or overexpressed, or on the contrary underproduced or underexpressed.

It is thus possible to measure characteristics such as efflux rate, which may provide information on the quantity of efflux pumps present at the bacterial membrane, or to measure the variation in pump activity (efflux capacity) following mutagenesis (induced or natural).

If the compound is stained and/or fluorescent, it will be possible to measure the coloration/fluorescence at the start of the experiment when the compound is brought into contact with the bacteria, and after a given time, either the fluorescence or the color in the bacteria, or in the external environment.

More advantageously, the invention relates to the above-mentioned use for assessing or measuring efflux by Gram-positive bacteria.

Multiple genes encoding efflux systems have been described in Gram-positive bacteria. Their product forms a protein transporter that reduces antibiotic accumulation in the cell.

For example, the msrA and msrB genes are responsible for an MS-type resistance phenotype, that is to say inducible resistance to macrolides with 14- and 15-carbon nuclei (C14 and C15) and to streptogramin B, after induction by erythromycin. The mef gene gives rise to a resistance phenotype known as M, characterized by limited resistance to C14 and C15 macrolides.

The vgaA and vgaB genes encode efflux proteins for synergistin A alone. All these genes are found in different species of Staphylococcus aureus (SA) and coagulase-negative staphylococci (CNS).

These are examples only, and should by no means be regarded as limiting the scope of the invention.

More advantageously, the invention relates to the above-mentioned use for assessing or measuring efflux by Gram-negative bacteria.

Advantageously, the above-mentioned use concerns the measurement or evaluation

In another aspect, the invention concerns a kit for detecting or measuring bacterial efflux, comprising
a compound as defined above, and
at least one control bacterium.

The kit of the invention therefore contains a compound according to the invention which is capable of being effluxed by bacteria, and additionally a control of the experiment, a control bacterium for which the efflux of said compound is known.

This control bacterium can express either a wild-type pump, the efflux of which is known precisely and reproducibly, or a mutant pump which no longer allows efflux due to a lack of expression of the proteins forming the pump or a structural modification affecting the function. Other control bacteria can of course be used, and in one advantageous aspect the kit can be supplied with both a positive control (a bacterium with a functional efflux pump) and a negative control (a bacterium with a non-functional or absent efflux pump).

Insofar as the compound according to the invention is stained and/or fluorescent, it is also possible to include in the kit according to the invention color palettes to define whether or not a bacterium is capable of effluxing a compound, or fluorescence detection means.

Advantageously, the invention relates to the use of the kit as defined above, for measuring or quantifying the efflux of said compound by a bacterium, in particular a Gram-positive bacterium, in particular S. aureus, B. subtilis and S. pneumoniae.

Advantageously, the invention concerns the use of the kit as defined above, for measuring or quantifying the efflux of said compound by a Gram-negative bacterium, in particular Escherichia coli.

Even more advantageously, the invention concerns the above-mentioned kit, additionally comprising a protonophore, notably chosen from 2,4-dinitrophenol, carbonyl cyanide-p-trifluoromethoxyphenylhydrazone (FCCP), carbonyl cyanide m-chlorophenyl hydrazone (CCCP), C4R1, ellipticine, 10-[2-(3-hydroxy-6-oxo-xanthen-9-yl)benzoyl]oxydecyl-triphenyl-phosphonium bromide and 2-(2-Hydroxyaryl)hexylphosphonium bromide, and in particular CCCP.

When measuring efflux from Gram-negative bacteria, the use of a protonophore, such as CCCP, is particularly appropriate. The protonophore in the above-mentioned kit is intended for use at concentrations ranging from 0.25 to 15 μg·mL⁻¹. This means that the protonophore can be used at any dose within this range, and in particular at a dose of 0.25 μg·mL⁻¹, 0.5 μg·mL⁻¹, 0.75 μg·mL⁻¹, 1 μg·mL⁻¹, 1.25 μg·mL⁻¹, 1.5 μg·mL⁻¹, 1.75 μg·mL⁻¹, 2 μg·mL⁻¹, 2.25 μg·mL⁻¹, 2.5 μg·mL⁻¹, 2.75 μg·mL⁻¹, 3 μg·mL⁻¹, 3.25 μg·mL⁻¹, 3.5 μg·mL⁻¹, 3.75 μg·mL⁻¹, 4 μg·mL⁻¹, 4.25 μg·mL⁻¹, 4.5 μg·mL⁻¹, 4.75 μg·mL⁻¹, 5 μg·mL⁻¹, 5.25 μg·mL⁻¹, 5.5 μg·mL⁻¹, 5.75 μg·mL⁻¹, 6 μg·mL⁻¹, 6.25 μg·mL⁻¹, 6.5 μg·mL⁻¹, 6.75 μg·mL⁻¹, 7 μg·mL⁻¹, 7.25 μg·mL⁻¹, 7.5 μg·mL⁻¹, 7.75 μg·mL⁻¹, 8 μg·mL⁻¹, 8.25 μg·mL⁻¹, 8.5 μg·mL⁻¹, 8.75 μg·mL⁻¹, 9 μg·mL⁻¹, 9.25 μg·mL⁻¹, 9.5 μg·mL⁻¹, 9.75 μg·mL⁻¹, 10 μg·mL⁻¹, 10.25 μg·mL⁻¹, 10.5 μg·mL⁻¹, 10.75 μg·mL⁻¹, 11 μg·mL⁻¹, 11.25 μg·mL⁻¹, 11.5 μg·mL⁻¹, 11.75 μg·mL⁻¹, 12 μg·mL⁻¹, 12.25 μg·mL⁻¹, 12.5 μg·mL⁻¹, 12.75 μg·mL⁻¹, 13 μg·mL⁻¹, 13.25 μg·mL⁻¹, 13.5 μg·mL⁻¹, 13.75 μg·mL⁻¹, 14 μg·mL⁻¹, 14.25 μg·mL⁻¹, 14.5 μg·mL⁻¹, 14.75 μg·mL⁻¹ or 15 μg·mL⁻¹. The person skilled in the art will be able to determine the appropriate dose depending on the protonophore used. For example, when using CCCP, it is advantageous to use a dose of 5 μg·mL⁻¹. In yet another aspect, the invention concerns the use of a compound as defined above, to assess the resistance of a bacterium to a bacteriostatic or bactericidal compound.

Since the compound according to the invention can be effluxed by bacterial pumps, it can logically be used to assess the efficiency of bacterial efflux.

If it is established that the compound according to the invention is effluxed, this means that efflux is functional and therefore that bacteria will be able to eliminate antibiotics. Thus, without the need to measure resistance using an antibiotic, we can conclude that the bacteria concerned are antibiotic-resistant via an efflux phenomenon.

Conversely, if the compound according to the invention is not effluxed, this means that the efflux pumps are not functional, and therefore antibiotics will not be effluxed either. So, in the same way as for resistance, it will be possible to determine easily, without using antibiotics, that bacteria are sensitive to said antibiotics due to lack of efflux.

In yet another aspect, the invention relates to a method for assessing resistance, in particular resistance due to an efflux mechanism, of a bacterium to a bacteriostatic or bactericidal compound, comprising:
cultivating bacteria in a suitable culture medium, to obtain a bacterial culture,
bringing the bacterial culture into contact with a compound mentioned above, to obtain a treated bacterial culture,
washing the treated bacterial culture, and precipitating the bacteria contained in the treated bacterial culture to obtain a bacterial pellet,
observing the staining of the bacterial pellet, such that
if the bacterial pellet has the color of a bacterial pellet from an untreated bacterial culture, said bacteria are resistant to said bacteriostatic or bactericidal compound, and
if the bacterial pellet has a color different from the color of a bacterial pellet from the untreated bacterial culture, said bacteria are not resistant to said bacteriostatic or bactericidal compound.

Alternatively, the evaluation method may include a filtration step to replace the centrifugation step. In this variant, the bacterial culture is run through a filter and the bacteria settle on it. The coloration of the solid deposit of bacteria present on the filter is then evaluated to determine whether said bacteria are resistant, notably due to an efflux mechanism, to a bacteriostatic or bactericidal compound. According to this embodiment, the invention thus relates to a method for assessing resistance, in particular resistance due to an efflux mechanism, of a bacterium to a bacteriostatic or bactericidal compound, comprising:
cultivating bacteria in a suitable culture medium, to obtain a bacterial culture,
bringing the bacterial culture into contact with a compound mentioned above, to obtain a treated bacterial culture,
washing the treated bacterial culture, and filtering the bacteria contained in the treated bacterial culture to obtain a filtrate on the one hand, and a bacterial deposit on a filter on the other,
observing the staining of the solid deposit on the filter, such that
if the solid deposit on the filter has the same color as a solid deposit from an untreated bacterial culture, said bacteria are resistant to said bacteriostatic or bactericidal compound, and
if the solid deposit has a color different from the color of a solid deposit from the untreated bacterial culture, said bacteria are not resistant to said bacteriostatic or bactericidal compound.

Advantageously, the invention relates to a method for assessing resistance, due to an efflux mechanism of a bacterium, to a bacteriostatic or bactericidal compound, comprising:
cultivating bacteria in a suitable culture medium, to obtain a bacterial culture,
bringing the bacterial culture into contact with a compound as defined above, to obtain a treated bacterial culture,
washing the treated bacterial culture, and precipitating the bacteria contained in the treated bacterial culture to obtain a bacterial pellet,
observing the staining of the bacterial pellet, such that
if the bacterial pellet has the color of a bacterial pellet from the untreated bacterial culture, said bacteria are resistant to said bacteriostatic or bactericidal compound, by efflux of said bacteriostatic or bactericidal compound, and
if the bacterial pellet has a color different from the color of a bacterial pellet from the untreated bacterial culture, said bacteria are not resistant to said bacteriostatic or bactericidal compound, by efflux of said bacteriostatic or bactericidal compound.

Even more advantageously, the invention relates to the above-mentioned method, said method further comprising a step of incubating the bacterial culture with a protonophore.

Advantageously, this means that the invention relates to a method for assessing resistance, due to an efflux mechanism of a bacterium, to a bacteriostatic or bactericidal compound, comprising:
cultivating bacteria in a suitable culture medium, to obtain a bacterial culture,
bringing the bacterial culture into contact with a protonophore, in particular a protonophore mentioned above, to obtain a treated bacterial culture,
bringing the pretreated bacterial culture into contact with a compound as defined above, to obtain a treated bacterial culture,
washing the treated bacterial culture, and precipitating the bacteria contained in the treated bacterial culture to obtain a bacterial pellet,
observing the staining of the bacterial pellet, such that
if the bacterial pellet has the color of a bacterial pellet from an untreated bacterial culture, said bacteria are resistant to said bacteriostatic or bactericidal compound, by efflux of said bacteriostatic or bactericidal compound, and
if the bacterial pellet has a color different from the color of a bacterial pellet from the untreated bacterial culture, said bacteria are not resistant to said bacteriostatic or bactericidal compound, by efflux of said bacteriostatic or bactericidal compound.

This pre-treatment method is particularly advantageous for measuring the efflux of Gram-negative bacteria. The above-mentioned compound is very simple and is based on the color properties of the compounds according to the invention.

By incubating bacteria with the compound according to the invention, the compound will accumulate in the bacteria.

If the efflux pumps are functional, the compound will be effluxed and released into the medium. The bacteria will no longer have any compounds, and the bacterial pellet resulting from the culture will have the color of a pellet that has not been treated with the colored compound.

Conversely, if the efflux pumps are not functional, the compound will accumulate in the bacteria, and the pellet will then take on a color which will be that (with some variation in intensity) of the colored compound.

A simple colorimetric test can be used, after washing to eliminate residual compounds that have been effluxed or not incorporated into the bacteria, to determine whether a bacterium is capable of effluxing compounds, and therefore whether or not it will be resistant to antibiotics.

Colorimetric tests can be carried out using techniques known to the person skilled in the art, and in particular according to the L*a*b* CIE 1976 color space. This color space is particularly common for characterizing surface colors. Three quantities characterize colors: the lightness L* derives from the luminance of the surface; the two parameters a* and b* express the deviation of the color from that of a gray surface of the same lightness.

### BRIEF DESCRIPTION OF THE FIGURES

The invention will be better understood on reading the following description given solely by way of example and with reference to the appended drawings in which:

FIG. 1 is a representative photograph of a test for NorA efflux pump activity in Staphylococcus aureus strains using compound 053 at a concentration of 0.75 μg·mL⁻¹. Pellet A represents a pellet of CIP 76.26, a reference strain with normal efflux activity—the pellet is pale pink. Pellet B represents a pellet of strain SA 1199, a clinical strain with mild to moderate efflux activity—the pellet is orange. Pellet C represents a pellet of strain SA 1199 B, a mutant strain with high efflux activity—the pellet is white/off-white. Pellet D represents a pellet from SA K1758, a mutant strain lacking an efflux pump—the pellet is bright dark pink.

FIG. 2 is a graph showing fluorescence intensity as a function of wavelength (in nm) for: A: buffer, B: strain SA K1758, C: strain CIP 76.26, D: strain SA 1199 B and E: strain SA K1758.

FIG. 3 is a histogram showing the maximum fluorescence emission values at 642 nm (after excitation at a wavelength of 530 nm) for: A: buffer, B: strain SA K1758, C: strain CIP 76.26, D: strain SA 1199 B and E: strain SA K1758.

FIG. 4 shows a representative photograph of a test using compound 053 at a concentration of 0.75 μg·mL⁻¹ on mutants of the Bacillus subtilis BmrA efflux pump. Pellet A represents a wild-type BmrA pellet—the pellet is pale pink. Pellet B represents a pellet from a strain overexpressing BmrA—the pellet is white. Pellet C represents a bacterial pellet with a BmrA deletion (BmrAΔ)—the pellet is dark pink.

FIG. 5 is a graph showing fluorescence intensity as a function of wavelength (in nm) for: A: buffer, B: BrmA-overexpressing B. subtilis strain, C: wild-type B. subtilis strain and D: BrmA A strain.

FIG. 6 is a histogram showing the maximum fluorescence emission values at 642 nm (after excitation at a wavelength of 530 nm) for: A: buffer, B: BrmA-overexpressing B. subtilis strain, C: wild-type B. subtilis strain and D: BrmA A strain.

FIG. 7 is a histogram showing the fluorescence intensity measured by flow cytometry for different strains of Staphylococcus aureus treated with compound 053. A: a wild-type strain corresponding to CIP 76.25, B: a reference strain corresponding to SA, C: a NorA-overproducing strain corresponding to SA1199B, and D: a NorA-deleted strain corresponding to SA K1758. ns: non-specific. * indicates a significant difference with a p-value of less than 0.05; ****=indicates a significant difference with a p-value of less than 0.001-One-way ANOVA test with Tukey test).

FIG. 8 is a histogram showing the relative fluorescence intensity (in arbitrary units) for each Staphylococcus aureus strain treated with compound 053 and fixed with PFA or ethanol. A: wild-type strain corresponding to CIP 76.25 fixed with PFA, B: reference strain corresponding to SA fixed with PFA, C: NorA overproducing strain corresponding to SA1199B fixed with PFA, D: NorA deleted strain corresponding to SA K1758 fixed with PFA, E: wild-type strain corresponding to CIP 76.25 fixed with ethanol, F: reference strain corresponding to SA fixed with ethanol, G: NorA overproducing strain corresponding to SA1199B fixed with ethanol, and H: NorA deleted strain corresponding to SA K1758 fixed with ethanol.

FIG. 9 shows a representative photograph of a test using compound 053 at a concentration of 1 μg·mL⁻¹ on mutants of the PatA-PatB transporter of Streptococcus pneumoniae. Pellet “9A” represents a wild-type PatA-PatB pellet. Pellet “9B” represents a pellet from a strain overexpressing PatA/PatB. Pellet “9C” represents a pellet from a strain with a PatA/PatB deletion.

FIG. 10 is a histogram showing the maximum fluorescence emission values at 642 nm (after excitation at a wavelength of 530 nm) for different strains of Streptococcus pneumoniae. A: wild-type PatA-PatB strain, B: strain overexpressing PatA/PatB, C: strain with PatA/PatB deletion.

FIG. 11 is a representative photograph of a test for AcrAB efflux pump activity in Escherichia coli strains using compound 392 at a concentration of 1 μg·mL⁻¹. Pellet “A” represents a pellet of strain ATCC11775, a reference strain with normal efflux activity. Pellet “B” represents a pellet of wild-type strain (AG100) with normal efflux activity. Pellet “C” represents a pellet of strain AG100A corresponding to a mutant strain deleted from the AcrAB efflux pump. Pellet “C” represents a pellet from AG102, a mutant strain overexpressing AcrAB.

FIG. 12 depicts a graph representative of a test for AcrAB efflux pump activity in Escherichia coli strains using compound 392 at a concentration of 1 μg·mL⁻¹. This graph shows fluorescence intensity as a function of wavelength (in nm). “A” represents strain ATCC11775, a reference strain with normal efflux activity. “B” represents the wild-type strain (AG100) with normal efflux activity. “C” represents strain AG100A corresponding to a mutant strain deleted from the AcrAB efflux pump. “D” represents a pellet from strain AG102, a mutant strain overexpressing AcrAB.

FIG. 13 is a histogram representative of a test for AcrAB efflux pump activity in Escherichia coli strains using compound 392 at a concentration of 1 μg·mL⁻¹. It shows as bars the maximum fluorescence emission values at 642 nm (after excitation at a wavelength of 530 nm) for different strains of Escherichia coli.). “A” represents strain ATCC11775, a reference strain with normal efflux activity. “B” represents the wild-type strain (AG100) with normal efflux activity. “C” represents strain AG100A corresponding to a mutant strain deleted from the AcrAB efflux pump. “D” represents a pellet from strain AG102, a mutant strain overexpressing AcrAB.

FIG. 14 is a photograph representative of a test for AcrAB efflux pump activity in Escherichia coli strains using compound 029 at a concentration of 1 μg·mL⁻¹. Pellet “A” represents a pellet of strain ATCC11775, a reference strain with normal efflux activity. Pellet “B” represents a pellet of wild-type strain (AG100) with normal efflux activity. Pellet “C” represents a pellet of strain AG100A corresponding to a mutant strain deleted from the AcrAB efflux pump. Pellet “D” represents a pellet from strain AG102, a mutant strain overexpressing AcrAB.

FIG. 15 shows a graph representative of a test for AcrAB efflux pump activity in Escherichia coli strains using compound 029 at a concentration of 1 μg·mL⁻¹. This graph shows fluorescence intensity as a function of wavelength (in nm). “A” represents strain ATCC11775, a reference strain with normal efflux activity. “B” represents the wild-type strain (AG100) with normal efflux activity. “C” represents strain AG100A corresponding to a mutant strain deleted from the AcrAB efflux pump. “D” represents a pellet from strain AG102, a mutant strain overexpressing AcrAB.

FIG. 16 is a histogram representative of a test for AcrAB efflux pump activity in Escherichia coli strains using compound 029 at a concentration of 1 μg·mL⁻¹. It shows as bars the maximum fluorescence emission values at 642 nm (after excitation at a wavelength of 530 nm) for different strains of Escherichia coli.). “A” represents strain ATCC11775, a reference strain with normal efflux activity. “B” represents the wild-type strain (AG100) with normal efflux activity. “C” represents strain AG100A corresponding to a mutant strain deleted from the AcrAB efflux pump. “D” represents a pellet from strain AG102, a mutant strain overexpressing AcrAB.

FIG. 17 is a photograph representative of a test for AcrAB efflux pump activity in Escherichia coli strains pretreated with Carbonyl Cyanide m-Chlorophenylhydrazone (CCCP) using compound 053 at a concentration of 1 μg·mL⁻¹. Pellet “A” represents a pellet of strain ATCC11775, a reference strain with normal efflux activity. Pellet “B” represents a pellet of wild-type strain (AG100) with normal efflux activity. Pellet “C” represents a pellet of strain AG100A corresponding to a mutant strain deleted from the AcrAB efflux pump. Pellet “D” represents a pellet from strain AG102, a mutant strain overexpressing AcrAB.

FIG. 18 is a graph representative of a test for AcrAB efflux pump activity in Escherichia coli strains pretreated with Carbonyl Cyanide m-Chlorophenylhydrazone using compound 053 at a concentration of 1 μg·ml⁻¹. This graph shows fluorescence intensity as a function of wavelength (in nm). “A” represents strain ATCC11775, a reference strain with normal efflux activity. “B” represents the wild-type strain (AG100) with normal efflux activity. “C” represents strain AG100A corresponding to a mutant strain deleted from the AcrAB efflux pump. “D” represents a pellet from strain AG102, a mutant strain overexpressing AcrAB.

FIG. 19 is a histogram representative of a test for AcrAB efflux pump activity in Escherichia coli strains pretreated with Carbonyl Cyanide m-Chlorophenylhydrazone using compound 053 at a concentration of 1 μg·mL⁻¹. It shows as bars the maximum fluorescence emission values at 642 nm (after excitation at a wavelength of 530 nm) for different strains of Escherichia coli.). “A” represents strain ATCC11775, a reference strain with normal efflux activity. “B” represents the wild-type strain (AG100) with normal efflux activity. “C” represents strain AG100A corresponding to a mutant strain deleted from the AcrAB efflux pump. “D” represents a pellet from strain AG102, a mutant strain overexpressing AcrAB.

### EXAMPLES

### Example 1—Synthesis

### 1. Generic Diagram for the Synthesis of Compounds According to the Invention

The synthesis diagram for the various compounds of the invention is given below. For three specific compounds, details of the synthesis including intermediate steps are also given below.

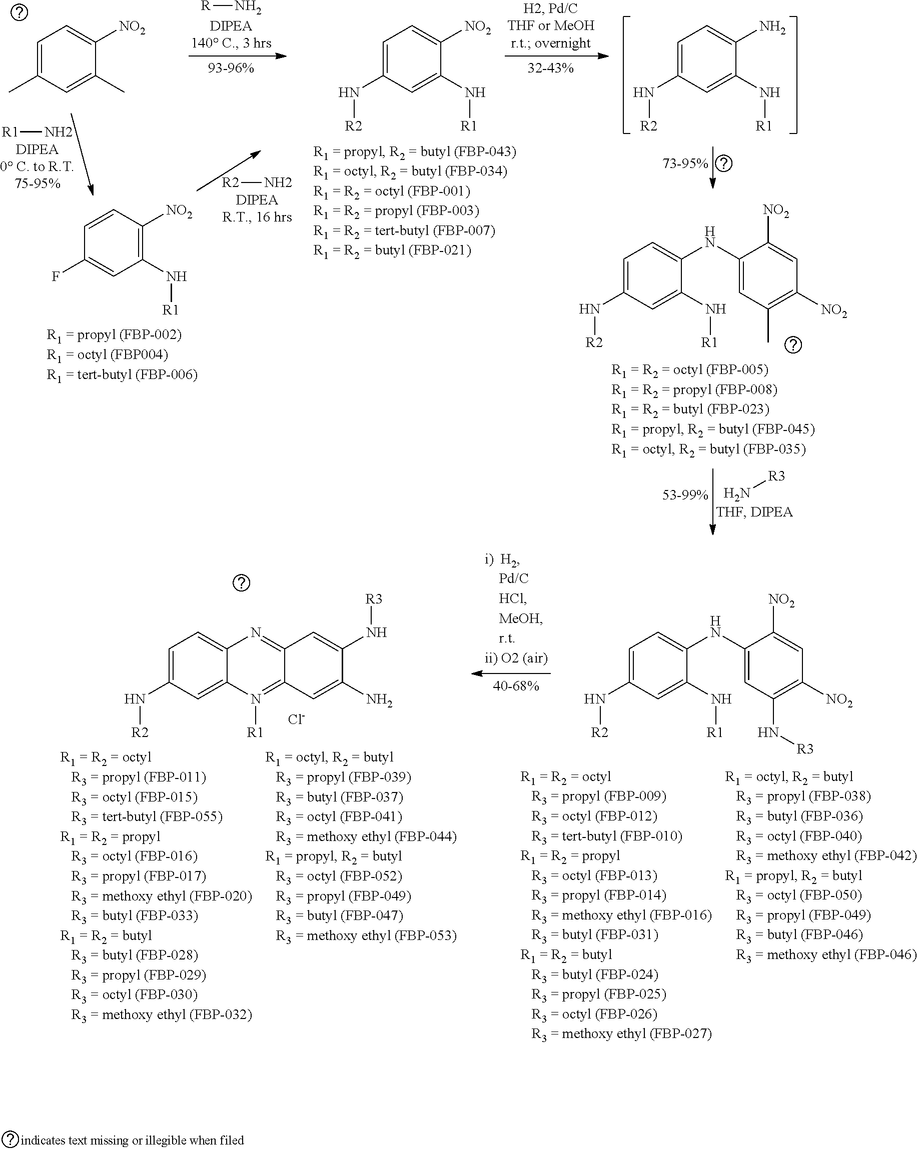

### 2—Synthesis of Compound 053

2a—Step 1

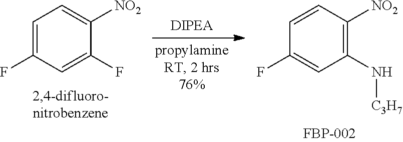

To a stirred solution of 1,4-dinitrofluorobenzene (DFNB-2 mL, 2.9 g, 18.23 mmol, 1 eq.) in dichloromethane (DCM-50 mL) at 0° C., propylamine (1.5 mL, 1.08 g, 18.23 mmol, 1 eq.) was added, followed by the addition of N, N-diisopropylethylamine (DIPEA-3.49 mL, 2.6 g, 20 mmol, 1.1 eq.). The reaction mixture was allowed to warm up to room temperature (over 30 min), after which the reaction was complete (monitored by thin-layer chromatography (TLC)). The solvent was evaporated in vacuo to give a yellow residue which was purified by SiO2 gel column chromatography to give the desired product FBP-002 as a yellow oil (2.74 g, 13.8 mmol, 76%).

¹H-NMR (400 MHZ; CDCl₃): δ 8.16 (m, 2H), 6.46 (dd, J₁=11.6 Hz, J₂=2.4 Hz, 1H), 6.34 (ddd, J₁=9.6 Hz, J₂=4.8 Hz, J₃=2.4 Hz, 1H), 3.21 (td, J₁=7 Hz, J₂=5.2 Hz, 2H), 1.75 (sext, J=7.3 Hz, 2H), 1.05 (t, J=7.4 Hz, 3H), ¹³C-NMR (101 MHZ; CDCl₃): δ 167.7 (d, J_{C-F}=257 Hz), 147.7 (d, J_{C-F}=14 Hz), 130.1 (d, J_{C-F}=13 Hz), 128.8, 103.9 (d, J_{C-F}=25 Hz), 99.3 (d, J_{C-F}=27 Hz), 45.1, 22.1, 11.6.

2b—Step 2

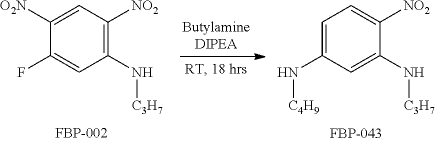

To a solution of FBP-002 (3 g, 15 mmol, 1.0 eq.) in tetrahydrofuran (THF −40 mL) were added butylamine (3.32 g, 4.48 mL, 45 mmol, 3 eq.) and DIPEA (3.3 g, 4.3 mL, 25.5 mmol, 1.7 eq.). The reaction mixture was stirred at 60° C. overnight. The solvent was then evaporated and the residue taken up in ethyl acetate (EtOAc-150 mL), washed with an aqueous solution of saturated ammonium chloride (2×150 mL) and brine (100 mL). The combined organic layers were then dried over MgSO₄, filtered and evaporated in vacuo. The crude obtained was purified by SiO2 gel column chromatography to give the desired product FBP-043 as an orange oil. (1.2 g, 4.8 mmol, 32% yield).

¹H-NMR (400 MHZ; CDCl₃): δ 8.16 (m, 2H), 6.46 (dd, J₁=11.6 Hz, J₂=2.4 Hz, 1H), 6.34 (ddd, J₁=9.6 Hz, J₂=4.8 Hz, J₃=2.4 Hz, 1H), 3.21 (td, J₁=7 Hz, J₂=5.2 Hz, 2H), 1.75 (sext, J=7.3 Hz, 2H), 1.05 (t, J=7.4 Hz, 3H), ¹³C-NMR (101 MHz; CDCl₃): δ 167.7 (d, J_{C-F}=257 Hz), 147.7 (d, J_{C-F}=14 Hz), 130.1 (d, J_{C-F}=13 Hz), 128.8, 103.9 (d, J_{C-F}=25 Hz), 99.3 (d, J_{C-F}=27 Hz), 45.1, 22.1, 11.6.

2c—Step 3

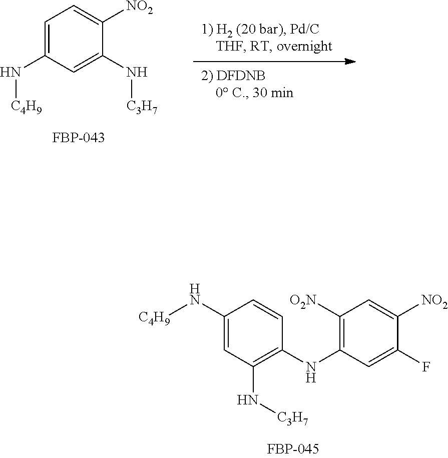

A solution of FBP-043 (1.174 g, 4.67 mmol, 1 eq.) in THF (70 mL) was hydrogenated (P=20 bar) in the presence of Pd/C (5%, 100 mg, 1.0% mol) overnight. After reducing the pressure, the solution was degassed by bubbling argon into the mixture. 1,5-difluoro-2,4-dinitrobenzene (905 mg, 4.4 mmol, 0.95 eq.) was added at 0° C. with stirring. Maintaining this condition for 20 min, the end of the reaction was detected by TLC (DCM/CH, 70/30). The Pd/Ca was then removed by filtration through a Celite® plug. The crude product was purified by silica gel column chromatography (DCM/EP, 50/50) to give the desired product FB-005 as a deep reddish solid (1.466 g, 3.62 mmol, 77% yield).

¹H-NMR (400 MHZ; CDCl₃): δ 9.30 (br, 1H), 9.14 (d, J=8 Hz, 1H), 6.82 (d, J=8.4 Hz, 1H), 6.53 (d, J=13.6 Hz, 1H), 5.99 (dd, J₁=8.4 Hz, J₂=2.4 Hz, 1H), 5.95 (d, J=2.4 Hz, 1H), 3.75 (br, 1H), 3.69 (br, 1H), 3.13 (t, J=6 Hz, 2H), 3.06 (q, J=6.4 Hz, 1H), 1.67-1.51 (m, 4H), 1.46 (sext, J=7.4 Hz, 2H), 0.97 (t, J=7.4 Hz, 3H), 0.92 (t, J=7.4 Hz, 3H), ¹³CRMN (101 MHz; CDCl₃): δ 159.9 (d, J_{C-F}=271 Hz), 150.5 (d, J_{C-F}=13 Hz), 150.3, 145.2, 128.7, 127.8, 126.9, 110.8, 103.9 (d, J_{C-F}=27 Hz), 101.7, 95.2, 45.3, 43.6, 43.2, 31.7, 22.6, 20.4, 14.0, 11.7.

1d—Step 4

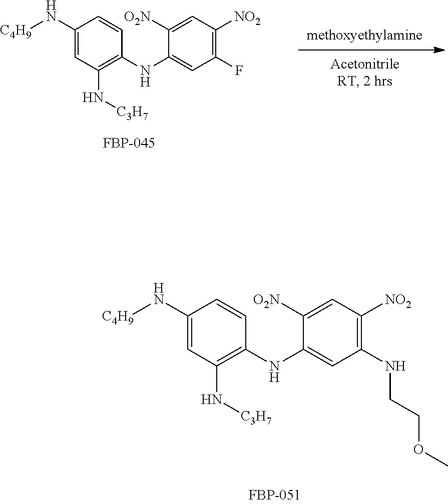

To a stirred solution of FB-045 (343 mg, 0.85 mmol, 1 eq.) in acetonitrile (15 mL), methoxyethylamine (221 μL, 191 mg, 2.54 mmol, 3 eq.) was added. The mixture was stirred at room temperature for 2 hours, when completion was monitored by TLC. After evaporation of the solvent, hot ethanol (10 mL) was added to the crude residue. A resulting asphalt-like material was collected by filtration and washed with hot water (30 mL), then recovered by dissolution in dichloromethane, dried over anhydrous MgSO₄ and filtered. After removing the solvent under reduced pressure and drying the solid in vacuo, the desired product FBP-051 was isolated as a dark asphalt-like solid (383 mg; 0.83 mmol; 98%).

¹H-NMR (400 MHZ; CDCl₃): δ 9.26 (s, 1H), 9.13 (br, 1H), 8.36 (t, J=4.6 Hz, 1H), 6.86 (d, J=8 Hz, 1H), 5.99 (dd, J₁=8.4 Hz, J₂=2.4 Hz, 1H), 5.95 (d, J=2.4 Hz, 1H), 5.71 (s, 1H), 3.79 (br, 1H), 3.67 (br, 1H), 3.54 (t, J=5.4 Hz, 2H), 3.35 (s, 3H), 3.19-3.10 (m, 4H), 3.06 (t, J=7 Hz, 2H), 1.67-1.51 (m, 4H), 1.45 (sext, J=7.4 Hz, 2H), 0.97 (t, J=7.4 Hz, 3H), 0.91 (t, J=7.4 Hz, 3H), ¹³CRMN (101 MHz; CDCl₃): δ 149.7, 149.3, 148.6, 145.4, 129.5, 128.8, 125.2, 124.5, 112.1, 101.6, 95.4, 93.2, 69.6, 59.1, 45.4, 43.8, 42.9, 31.8, 22.7, 20.4, 14.0, 11.7.

2e—Step 5

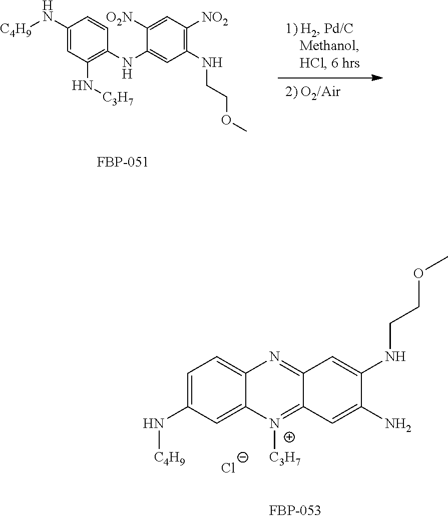

A solution of compound FBP-051 (433 mg, 0.94 mmol, 1 equiv.) in methanol (20 mL) was hydrogenated (20 bar) in the presence of Pd/C (5 wt. %, 36 mg) and HCl (12M, 0.30 mL) for 6 hrs. The mixture was then stirred in air for 16 h. The Pd/Ca was removed by filtration through a Celite® plug, which was rinsed several times with methanol and dichloromethane. After removal of the solvent under reduced pressure, the resulting solid was taken up with dichloromethane (50 mL), and extracted with brine (2×50 mL), the organic layer was then dried over MgSO₄, filtered and evaporated in vacuo to give a crude black solid which was then purified on alumina gel chromatography column (DCM/MeOH) and led to a deep purple solid of compound FBP-053 or compound 053 (177 mg, 0.44 mmol, 49% yield).

¹H-NMR (400 MHZ; CD₃OD): δ 7.68 (d, J=9.2 Hz, 1H), 7.17 (dd, J₁=9.2 Hz, J₂=1.6 Hz, 1H), 6.94 (d, J=1.6 Hz, 1H), 6.78 (s, 1H), 6.52 (br, 1H), 4.52 (t, J=8 Hz, 2H), 3.73 (t, J=5.2 Hz, 2H), 3.45 (t, J=5.2 Hz, 2H), 3.42 (s, 3H), 3.32 (t, J=7.2 Hz, 2H), 3.29 (sext, J=1.6 Hz, 2H), 1.96 (sext, J=7.6 Hz, 2H), 1.71 (quint, J=7 Hz, 2H), 1.51 (sext, J=7 Hz, 2H), 1.19 (t, J=7.4 Hz, 3H), 1.01 (t, J=7.4 Hz, 3H), ¹³C-NMR (101 MHz; CD₃OD): δ 150.9, 139.2, 138.4, 135.5, 132.9, 131.4, 130.9, 92.4, 69.9, 57.8, 43.3, 42.7, 30.5, 20.1, 19.7, 12.9, 10.1; 5 masked or overlapping signals. High-resolution mass spectrometry: calculated for C₂₂H₃₂N₅O⁺: 382.2601; obtained: 382.2593

### 3—Synthesis of Compound 052

3a—Step 1

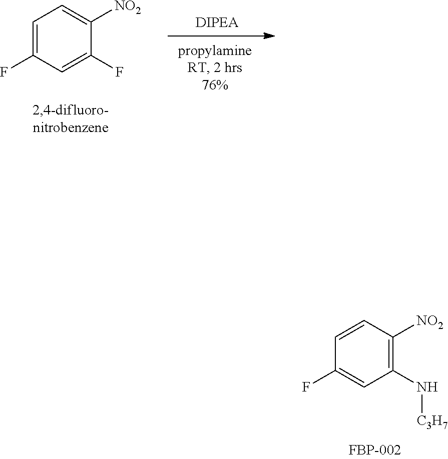

To a stirred solution of DFNB (2 mL, 2.9 g, 18.23 mmol, 1 eq.) in DCM (50 mL) at 0° C., propylamine (1.5 mL, 1.08 g, 18.23 mmol, 1 eq.) was added, followed by the addition of DIPEA (3.49 mL, 2.6 g, 20 mmol, 1.1 eq.). The reaction mixture was allowed to warm up to room temperature (over 30 min), after which the reaction was complete (monitored by TLC). The solvent was evaporated in vacuo to give a yellow residue which was purified by SiO2 gel column chromatography to give the desired product FBP-002 as a yellow oil (2.74 g, 13.8 mmol, 76%).

¹H-NMR (400 MHZ; CDCl₃): δ 8.16 (m, 2H), 6.46 (dd, J₁=11.6 Hz, J₂=2.4 Hz, 1H), 6.34 (ddd, J₁=9.6 Hz, J₂=4.8 Hz, J₃=2.4 Hz, 1H), 3.21 (td, J₁=7 Hz, J₂=5.2 Hz, 2H), 1.75 (sext, J=7.3 Hz, 2H), 1.05 (t, J=7.4 Hz, 3H).

¹³C-NMR (101 MHZ; CDCl₃): δ 167.7 (d, J_{C-F}=257 Hz), 147.7 (d, J_{C-F}=14 Hz), 130.1 (d, J_{C-F}=13 Hz), 128.8, 103.9 (d, J_{C-F}=25 Hz), 99.3 (d, J_{C-F}=27 Hz), 45.1, 22.1, 11.6.

3b—Step 2

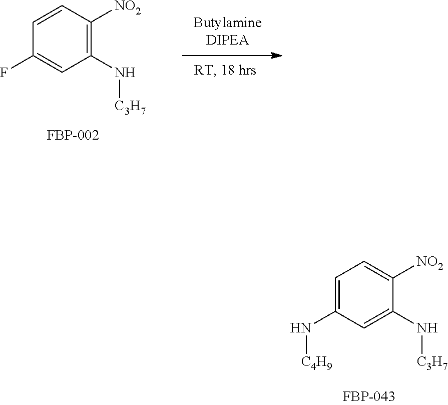

To a solution of FBP-002 (3 g, 15 mmol, 1.0 eq.) in THF (40 mL) were added butylamine (3.32 g, 4.48 mL, 45 mmol, 3 eq.) and DIPEA (3.3 g, 4.3 mL, 25.5 mmol, 1.7 eq.). The reaction mixture was stirred at 60° C. overnight. The solvent was then evaporated and the residue taken up in EtOAc (150 mL), washed with an aqueous solution of saturated ammonium chloride (2×150 mL) and brine (100 mL). The combined organic layers were then dried over MgSO₄, filtered and evaporated in vacuo. The crude obtained was purified by SiO2 gel column chromatography to give the desired product FBP-043 as an orange oil. (1.2 g, 4.8 mmol, 32% yield).

¹H-NMR (400 MHZ; CDCl₃): δ 8.52 (br, 1H), 7.99 (d, J=9.2 Hz, 1H), 5.89 (d, J=9.6 Hz, 1H), 5.61 (s, 1H), 4.41 (br, 1H), 3.18 (m, 4H), 1.75 (sext, J=7.2 Hz, 2H), 1.62 (quint, J=7.2 Hz, 2H), 1.43 (sext, J=7.5 Hz, 2H), 1.04 (t, J=7.4 Hz, 3H), 0.96 (t, J=7.2 Hz, 3H).

¹³C-NMR (101 MHZ; CDCl₃): δ 154.5, 148.7, 129.3, 123.7, 104.8, 90.0, 44.8, 43.0, 31.3, 22.2, 20.3, 13.9, 11.8.

3c—Step 3

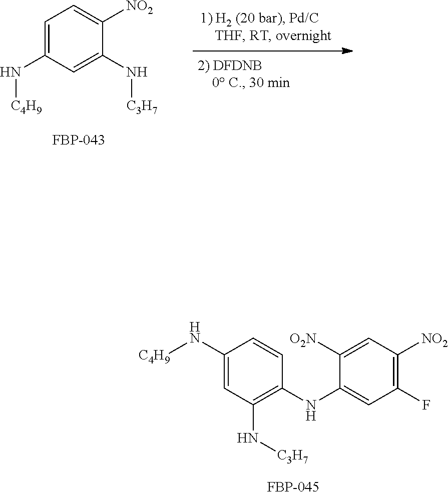

A solution of FBP-043 (1.174 g, 4.67 mmol, 1 eq.) in THF (70 mL) was hydrogenated (P=20 bar) in the presence of Pd/C (5%, 100 mg, 1.0% mol) overnight. After reducing the pressure, the solution was degassed by bubbling Ar into the mixture. 1,5-difluoro-2,4-dinitrobenzene (905 mg, 4.4 mmol, 0.95 eq.) was added at 0° C. with stirring. Maintaining this condition for 20 min, the end of the reaction was detected by TLC (DCM/CH, 70/30). The Pd/Ca was then removed by filtration through a Celite® plug. The crude product was purified by silica gel column chromatography (DCM/EP, 50/50) to give the desired product FB-045 as a deep reddish solid (1.466 g, 3.62 mmol, 77% yield).

¹H-NMR (400 MHZ; CDCl₃): δ 9.30 (br, 1H), 9.14 (d, J=8 Hz, 1H), 6.82 (d, J=8.4 Hz, 1H), 6.53 (d, J=13.6 Hz, 1H), 5.99 (dd, J₁=8.4 Hz, J₂=2.4 Hz, 1H), 5.95 (d, J=2.4 Hz, 1H), 3.75 (br, 1H), 3.69 (br, 1H), 3.13 (t, J=6 Hz, 2H), 3.06 (q, J=6.4 Hz, 1H), 1.67-1.51 (m, 4H), 1.46 (sext, J=7.4 Hz, 2H), 0.97 (t, J=7.4 Hz, 3H), 0.92 (t, J=7.4 Hz, 3H).

¹³C-NMR (101 MHz; CDCl₃): δ 159.9 (d, J_{C-F}=271 Hz), 150.5 (d, J_{C-F}=13 Hz), 150.3, 145.2, 128.7, 127.8, 126.9, 110.8, 103.9 (d, J_{C-F}=27 Hz), 101.7, 95.2, 45.3, 43.6, 43.2, 31.7, 22.6, 20.4, 14.0, 11.7.

3d—Step 4

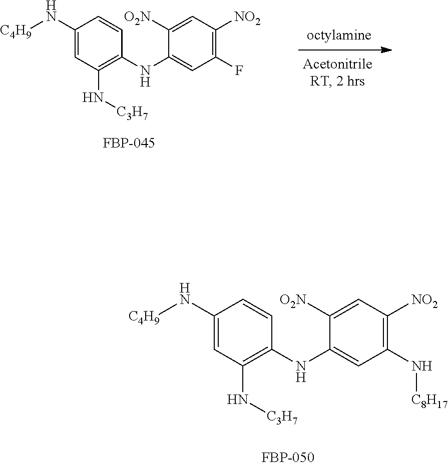

To a stirred solution of FB-045 (327 mg, 0.81 mmol, 1 eq.) in acetonitrile (15 mL), octylamine (398 μL, 311 mg, 2.41 mmol, 3 eq.) was added. The mixture was stirred at room temperature for 2 hours, when completion was monitored by TLC. After evaporation of the solvent, hot ethanol (10 mL) was added to the crude residue. A resulting asphalt-like material was collected by filtration and washed with hot water (30 mL), then recovered by dissolution in dichloromethane, dried over anhydrous MgSO₄ and filtered. After removing the solvent under reduced pressure and drying the solid in vacuo, the desired product FBP-050 was isolated as a dark solid (392 mg; 0.76 mmol; yield 94%).

¹H-NMR (400 MHZ; CDCl₃): δ 9.26 (s, 1H), 9.12 (s, 1H), 8.20 (t, J=5 Hz, 1H), 6.86 (d, J=8.4 Hz, 1H), 5.99 (dd, J₁=8.2 Hz, J₂=2.4 Hz, 1H), 5.95 (d, J=2.4 Hz, 1H), 5.69 (s, 1H), 3.79 (t, J=5 Hz, 1H), 3.65 (br, 1H), 3.13 (t, J=7 Hz, 2H), 3.06 (q, J=6.3 Hz, 2H), 2.99 (q, J=6.3 Hz, 2H), 1.97-1.50 (m, 8H), 1.45 (sext, J=8 Hz, 2H), 1.30 (m, 8H), 0.97 (t, J=7.2 Hz, 3H), 0.91 (t, J=7.4 Hz, 3H), 0.87 (t, J=7.4 Hz, 3H).

¹³C-NMR (101 MHz; CDCl₃): δ 149.7, 149.2, 148.6, 129.6, 128.8, 125.0, 124.5, 112.1, 101.6, 95.3, 93.1, 45.4, 43.9, 43.2, 31.9, 31.8, 29.3, 29.2, 28.4, 27.0, 22.7, 22.6, 20.4, 14.2, 14.0, 13.9, 11.7.

3e—Step 5

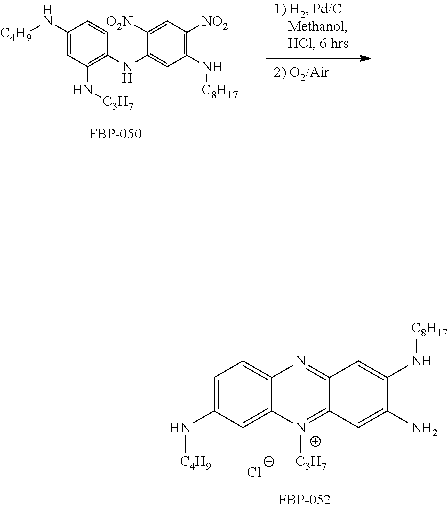

A solution of compound FBP-050 (316 mg, 0.61 mmol, 1 equiv.) in methanol (20 mL) was hydrogenated (20 bar) in the presence of Pd/C (5 wt. %, 24 mg) and HCl (12M, 0.25 mL) for 6 hrs. The mixture was then stirred in air for 16 h. The Pd/Ca was removed by filtration through a Celite® plug, which was rinsed several times with methanol and dichloromethane. After removal of the solvent under reduced pressure, the resulting solid was taken up with dichloromethane (50 mL), and extracted with brine (2×50 mL), the organic layer was then dried over MgSO₄, filtered and evaporated in vacuo to give a crude black solid which was then purified on alumina gel chromatography column (DCM/MeOH) and led to a deep purple solid of compound FBP-052 or compound 052 (129 mg, 0.27 mmol, 45% yield).

¹H-NMR (400 MHZ; CD₃OD): δ 7.72 (d, J=9.6 Hz, 1H), 7.18 (dd, J1=9.2 Hz, J₂=2 Hz, 1H), 6.96 (s, 1H), 6.75 (s, 1H), 6.56 (br, 1H), 4.55 (t, J=8.2 Hz, 2H), 3.32 (m, 2H), 3.28 (quint, J=1.6 Hz, 2H), 3.25 (t, J=7.4 Hz, 2H), 1.98 (sext, J=8 Hz, 2H), 1.80-1.66 (m, 4H), 1.49 (quint, J=8 Hz, 4H), 1.41-1.26 (m, 8H), 1.20 (t, J=7.4 Hz, 3H), 1.01 (t, J=7.4 Hz, 3H), 0.88 (t, J=7.4 Hz, 3H).

¹³C-NMR (101 MHz; CDCl₃): δ 153.3, 150.9, 139.2, 138.6, 135.4, 131.3, 130.9, 102.8, 92.4, 48.9, 43.7, 42.7, 31.7, 30.5, 29.3, 29.1, 28.1, 27.1, 22.4, 20.1, 19.7, 13.1, 12.9, 10.1. 3 signals are masked or overlapping.

### 4—Synthesis of Compound 047

4a—Step 1

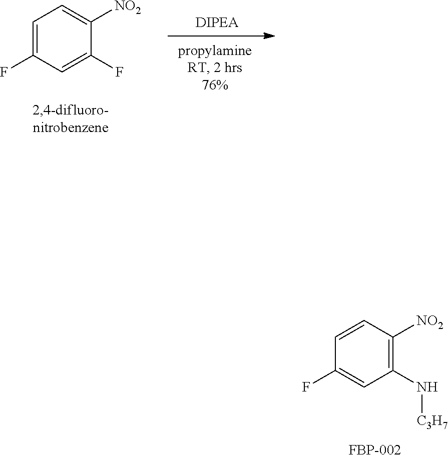

To a stirred solution of 1,4-dinitrofluorobenzene (DFNB-2 mL, 2.9 g, 18.23 mmol, 1 eq.) in dichloromethane (DCM-50 mL) at 0° C., propylamine (1.5 mL, 1.08 g, 18.23 mmol, 1 eq.) was added, followed by the addition of N, N-diisopropylethylamine (DIPEA-3.49 mL, 2.6 g, 20 mmol, 1.1 eq.). The reaction mixture was allowed to warm up to room temperature (over 30 min), after which the reaction was complete (monitored by thin-layer chromatography (TLC)). The solvent was evaporated in vacuo to give a yellow residue which was purified by SiO2 gel column chromatography to give the desired product FBP-002 as a yellow oil (2.74 g, 13.8 mmol, 76%).

¹H-NMR (400 MHZ; CDCl₃): δ 8.16 (m, 2H), 6.46 (dd, J₁=11.6 Hz, J₂=2.4 Hz, 1H), 6.34 (ddd, J₁=9.6 Hz, J₂=4.8 Hz, J₃=2.4 Hz, 1H), 3.21 (td, J₁=7 Hz, J₂=5.2 Hz, 2H), 1.75 (sext, J=7.3 Hz, 2H), 1.05 (t, J=7.4 Hz, 3H), ¹³C-NMR (101 MHz; CDCl₃): δ 167.7 (d, J_{C-F}=257 Hz), 147.7 (d, J_{C-F}=14 Hz), 130.1 (d, J_{C-F}=13 Hz), 128.8, 103.9 (d, J_{C-F}=25 Hz), 99.3 (d, J_{C-F}=27 Hz), 45.1, 22.1, 11.6.

4b—Step 2

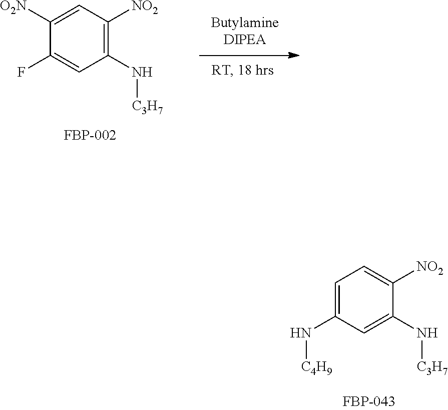

To a solution of FBP-002 (3 g, 15 mmol, 1.0 eq.) in tetrahydrofuran (THF −40 mL) were added butylamine (3.32 g, 4.48 mL, 45 mmol, 3 eq.) and DIPEA (3.3 g, 4.3 mL, 25.5 mmol, 1.7 eq.). The reaction mixture was stirred at 60° C. overnight. The solvent was then evaporated and the residue taken up in ethyl acetate (EtOAc-150 mL), washed with an aqueous solution of saturated ammonium chloride (2×150 mL) and brine (100 mL). The combined organic layers were then dried over MgSO₄, filtered and evaporated in vacuo. The crude obtained was purified by SiO2 gel column chromatography to give the desired product FBP-043 as an orange oil. (1.2 g, 4.8 mmol, 32% yield).

¹H-NMR (400 MHZ; CDCl₃): δ 8.16 (m, 2H), 6.46 (dd, J₁=11.6 Hz, J₂=2.4 Hz, 1H), 6.34 (ddd, J₁=9.6 Hz, J₂=4.8 Hz, J₃=2.4 Hz, 1H), 3.21 (td, J₁=7 Hz, J₂=5.2 Hz, 2H), 1.75 (sext, J=7.3 Hz, 2H), 1.05 (t, J=7.4 Hz, 3H), ¹³C-NMR (101 MHz; CDCl₃): δ 167.7 (d, J_{C-F}=257 Hz), 147.7 (d, J_{C-F}=14 Hz), 130.1 (d, J_{C-F}=13 Hz), 128.8, 103.9 (d, J_{C-F}=25 Hz), 99.3 (d, J_{C-F}=27 Hz), 45.1, 22.1, 11.6.

4c—Step 3

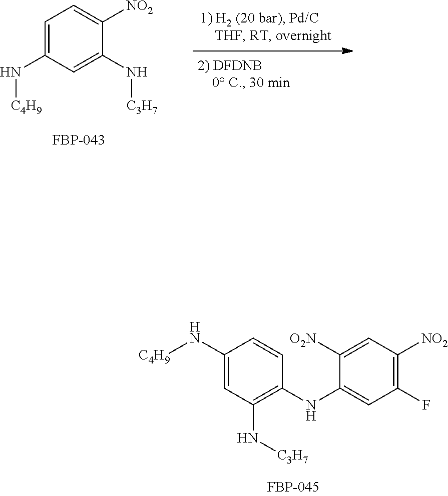

A solution of FBP-043 (1.174 g, 4.67 mmol, 1 eq.) in THF (70 mL) was hydrogenated (P=20 bar) in the presence of Pd/C (5%, 100 mg, 1.0% mol) overnight. After reducing the pressure, the solution was degassed by bubbling argon into the mixture. 1,5-difluoro-2,4-dinitrobenzene (905 mg, 4.4 mmol, 0.95 eq.) was added at 0° C. with stirring. Maintaining this condition for 20 min, the end of the reaction was detected by TLC (DCM/CH, 70/30). The Pd/Ca was then removed by filtration through a Celite® plug. The crude product was purified by silica gel column chromatography (DCM/EP, 50/50) to give the desired product FB-005 as a deep reddish solid (1.466 g, 3.62 mmol, 77% yield).

¹H-NMR (400 MHZ; CDCl₃): δ 9.30 (br, 1H), 9.14 (d, J=8 Hz, 1H), 6.82 (d, J=8.4 Hz, 1H), 6.53 (d, J=13.6 Hz, 1H), 5.99 (dd, J₁=8.4 Hz, J₂=2.4 Hz, 1H), 5.95 (d, J=2.4 Hz, 1H), 3.75 (br, 1H), 3.69 (br, 1H), 3.13 (t, J=6 Hz, 2H), 3.06 (q, J=6.4 Hz, 1H), 1.67-1.51 (m, 4H), 1.46 (sext, J=7.4 Hz, 2H), 0.97 (t, J=7.4 Hz, 3H), 0.92 (t, J=7.4 Hz, 3H), ¹³CRMN (101 MHz; CDCl₃): δ 159.9 (d, J_{C-F}=271 Hz), 150.5 (d, J_{C-F}=13 Hz), 150.3, 145.2, 128.7, 127.8, 126.9, 110.8, 103.9 (d, J_{C-F}=27 Hz), 101.7, 95.2, 45.3, 43.6, 43.2, 31.7, 22.6, 20.4, 14.0, 11.7.

4d—Step 4

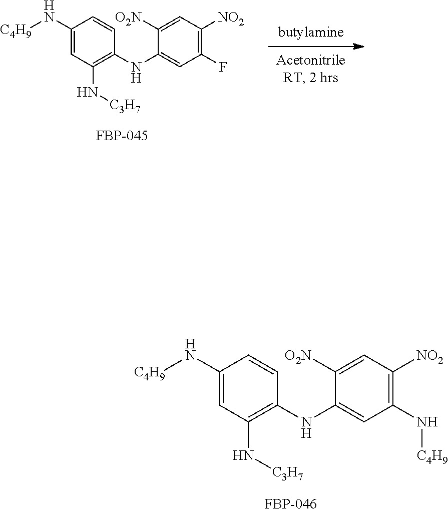

To a stirred solution of FB-045 (341 mg, 0.7 mmol, 0.84 mmol, 1 eq.) in acetonitrile (15 mL), butylamine (249 μL, 186 mg, 2.52 mmol, 3 eq.) was added. The mixture was stirred at room temperature for 2 hours, when completion was monitored by TLC. After evaporation of the solvent, hot ethanol (10 mL) was added to the crude residue. A resulting asphalt-like material was collected by filtration and washed with hot water (30 mL), then recovered by dissolution in dichloromethane, dried over anhydrous MgSO₄ and filtered. After removing the solvent under reduced pressure and drying the solid in vacuo, the desired product was isolated as a dark solid (354 mg; 0.67 mmol; 96%).

¹H-NMR (400 MHZ; CDCl₃): δ 9.26 (s, 1H), 9.11 (s, 1H), 8.20 (t, J=5 Hz, 1H), 6.86 (d, J=8.4 Hz, 1H), 5.98 (dd, J₁=8.4 Hz, J₂=2.4 Hz, 1H), 5.96 (d, J=2.4 Hz, 1H), 5.69 (s, 1H), 3.80 (t, J=5.2 Hz, 1H), 3.67 (br, 1H), 3.13 (t, J=7 Hz, 2H), 3.06 (q, J=6.7 Hz, 2H), 3.01 (q, J=6.7 Hz, 2H), 3.06 (t, J=7 Hz, 2H), 1.67-1.51 (m, 4H), 1.45 (sext, J=7.4 Hz, 2H), 1.33 (sext, J=7.4 Hz, 2H), 0.97 (t, J=7.4 Hz, 3H), 0.91 (t, J=7.4 Hz, 3H), 0.88 (t, J=7.4 Hz, 3H).

¹³C-NMR (101 MHZ; CDCl₃): δ 149.7, 149.2, 148.6, 145.4, 129.6, 128.8, 125.0, 124.5, 112.1, 101.6, 95.3, 93.1, 45.4, 43.9, 42.9, 31.8, 30.4, 22.7, 20.4, 20.2, 14.0, 13.8, 11.7.

4e—Step 5

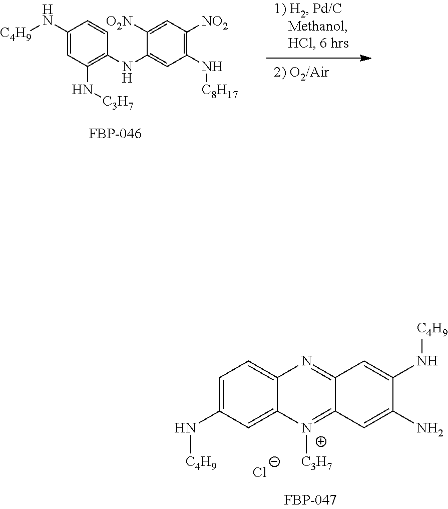

A solution of compound FBP-046 (340 mg, 0.74 mmol, 1 equiv.) in methanol (20 mL) was hydrogenated (20 bar) in the presence of Pd/C (5 wt. %, 29 mg) and HCl (12M, 0.3 mL) for 6 hours. The mixture was then stirred in air for 16 hours. Pd/Ca was removed by filtration through a Celite® plug, which was rinsed several times with methanol and dichloromethane. After removal of the solvent under reduced pressure, the resulting solid was taken up with dichloromethane (50 mL), and extracted with brine (2×50 mL), the organic layer was then dried over MgSO₄, filtered and evaporated in vacuo to give a crude black/purple solid which was then purified on alumina gel chromatography column (DCM/MeOH) and led to a deep purple solid of compound FBP-047 or compound 047 (130 mg, 0.31 mmol, 42% yield).

¹H-NMR (400 MHZ; CD₃OD): δ 7.72 (d, J=9.2 Hz, 1H), 7.19 (dd, J₁=9.2 Hz, J₂=2 Hz, 1H), 6.97 (s, 1H), 6.76 (s, 1H), 6.5 (br, 1H), 4.55 (t, J=8 Hz, 2H), 3.35-3.24 (m, 8H), 1.98 (sext, J=7.6 Hz, 2H), 1.80-1.64 (m, 4H), 1.51 (quint, J=7.2 Hz, 4H), 1.20 (t, J=7.4 Hz, 3H), 1.02 (t, J=7.4 Hz, 3H), 1.01 (t, J=7.4 Hz, 3H).

¹³C-NMR (101 MHZ; CD₃OD): δ 153.3, 150.8, 139.2, 138.5, 135.3, 132.6, 131.3, 130.8, 119.7, 102.7, 92.4, 89.3, 48.9, 43.4, 42.8, 30.5, 30.2, 20.3, 20.1, 19.7, 13.0, 10.1. 1 signal is masked or overlapping.

### 5—Synthesis of Compound 029

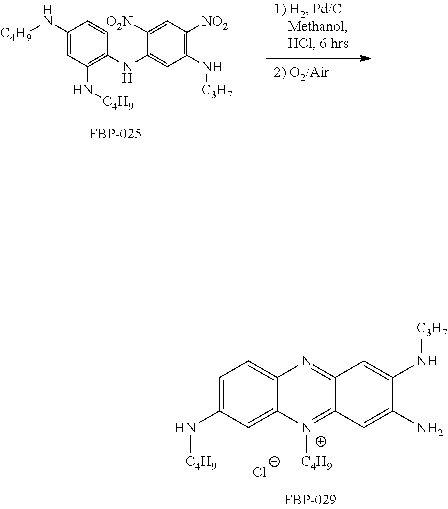

A solution of compound FBP-025 (356 mg, 0.78 mmol, 1 equiv.) in methanol (20 mL) was hydrogenated (20 bar) in the presence of Pd/C (5 wt. %, 30 mg) and HCl (12M, 0.3 mL) for 6 hrs. The mixture was then stirred overnight in air. Pd/Ca was removed by filtration through a Celite® plug, which was rinsed several times with methanol and dichloromethane. After removal of the solvent under reduced pressure, the resulting solid was taken up with dichloromethane (50 mL), and extracted with brine (2×50 mL), the organic layer was then dried over MgSO₄, filtered and evaporated in vacuo to give a crude black solid which was then purified on an alumina gel chromatography column (Brockman activity I) (DCM/MeOH) and led to a deep purple solid (160 mg, 39 mmol; 50%).

¹H-NMR (400 MHZ; CD₃OD): δ 7.68 (d, J=9.2 Hz, 1H), 7.16 (dd, J₁=9.2 Hz, J₂=2 Hz, 1H), 6.93 (s, 1H), 6.70 (s, 1H), 6.5 (br, 1H), 4.54 (t, J=8 Hz, 2H), 3.32-3.19 (m, 8H), 1.89 (m, 2H), 1.79 (quint, J=7.4 Hz, 2H), 1.73-1.58 (m, 4H), 1.49 (quint, J=7.5 Hz, 2H), 1.1 (t, J=7.2 Hz, 3H), 1.08 (t, J=7.4 Hz, 3H), 1.01 (t, J=7.4 Hz, 3H).

¹³C-NMR (101 MHZ; CD₃OD): δ 153.3, 150.8, 139.2, 138.5, 135.3, 132.6, 131.3, 130.8, 119.7, 102.7, 92.4, 89.3, 48.9, 43.4, 43.3, 42.6, 30.5, 30.2, 20.3, 20.2, 19.7, 13.0, 10.2.

### 6—Synthesis of Compound 392 or 44

A solution of compound TM390 (3.08 g, 5.8 mmol, 1 equiv.) in methanol (60 mL) was hydrogenated (40 bar) in the presence of Pd/C (5 wt. %, 617 mg) and HCl (12M, 1 mL) for 16 hrs. The mixture was then stirred in air for 16 hrs. The Pd/Ca was removed by filtration through a Celite® plug, which was rinsed several times with methanol and dichloromethane. After removal of the solvent under reduced pressure, the resulting solid was purified on an alumina gel chromatography column (DCM/MeOH95:5->90:10) and led to TM 392 as a deep purple solid (3% yield).

¹H-NMR (400 MHZ; CD₃OD): δ 7.56 (d, ³J=8.97 Hz, 1H), 7.11 (dd, ³J=9.25 Hz, 4J=1.87 Hz, 1H), 6.84 (s, 1H), 6.60 (s, 1H), 6.34 (br, 1H), 4.44 (t, ³J=7.99 Hz, 2H), 3.76 (t, 3J=5.29 Hz, 2H), 3.44 (s, 3H), 3.41 (t, 3J=5.41 Hz, 2H), 3.27 (t, 3J=7.09 Hz, 2H), 1.85-1.32 (mm, 16H), 1.04 (t, 3J=7.2 Hz, 3H), 0.92 (t, 3J=7.08 Hz, 3H).

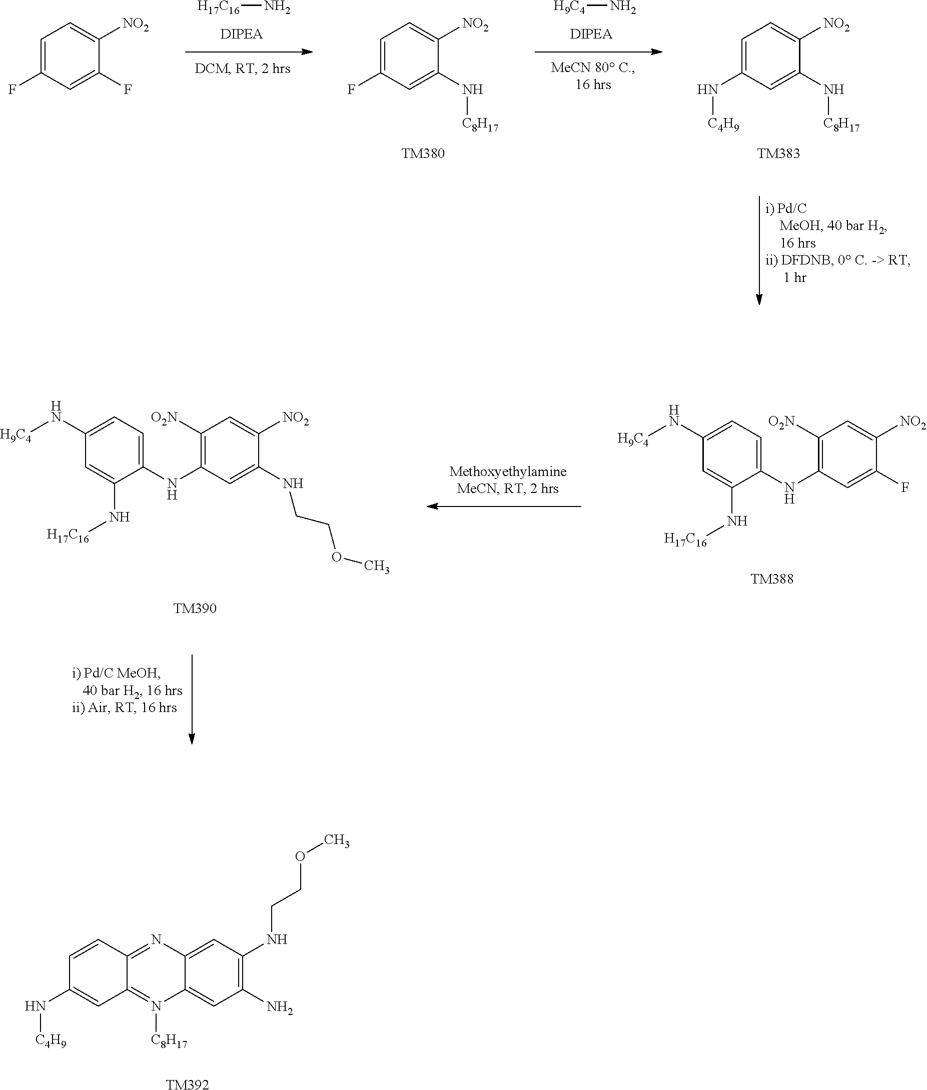

### Example 2—Identification of Antibiotic Efflux and Quantification of its Efficacy

Antibiotic efflux is a phenomenon recognized as extremely serious by health authorities worldwide. It concerns all bacteria, but is obviously of greatest concern in hospital settings, such as intensive care, orthopedic and thoracic surgery, and diabetes-associated infections.

Antibiotic efflux, by virtue of its poly-specificity, has long been identified as a complement to more conventional resistance mechanisms (target mutation, enzymes such as Beta-lactamase, etc.), but more recently, as intervening upstream of the other mechanisms, encouraging their selection.

While there are methods for identifying the main resistance mechanisms in biology laboratories, efflux is not identified or identifiable by any standardized method except by deduction, which is not always very reliable.

It is therefore necessary to develop a method for identifying antibiotic efflux and quantifying its efficacy. Compound 053's staining and fluorescence characteristics made it an excellent tool for this type of project.

### Example 2-1: Staphylococcus aureus

### Materials and Methods

Bacterial strains: The inventors used the following strains of Staphylococcus aureus:
CIP 76.25, a reference strain producing a basal level of NorA,
SA1199, a reference strain producing a basal level of NorA,
SA 1199B, an overproducer of the NorA pump, and
SA K1758 with the NorA gene deleted, (Kaatz et al., 2003, Antimicrob Agents Chemother. 2003 February; 47(2): 719-26).

Compound: Compound 053 (see synthesis in example 1) is prepared in pure DMSO (Sigma) at a concentration of 20 mg/mL. It is then diluted in milliQ water to obtain a stock solution of 1000 μg/mL.

Bacterial staining: Bacteria in exponential growth phase are incubated for 15 min in the presence of compound 053. The bacteria are then washed twice in phosphate buffer and centrifuged, with the pellets observed with the naked eye.

Fluorescence measurement: Bacteria in exponential growth phase are incubated for 15 min in the presence of compound 053. The optical density at 600 nm is then adjusted to 0.5 and 50 μL is transferred to a black microtiter plate. Fluorescence is read in a Tecan M200 microplate reader.

The following protocol was followed:
1. Grow bacteria on a freshly prepared Muller Hilton-II (MH-II) agar plate. Incubate the Petri dish overnight at 37° C. (Note: Incubation temperature and growth time depend on the bacteria used in the experiment).
2. The next morning, pick one or two colonies from the overnight plate and inoculate them in an Erlenmeyer flask containing 20 mL MH-II broth.
3. Incubate the Erlenmeyer on an orbital shaker at 37° C. and 160 rpm for 3-4 hrs. Note: This step allows us to obtain cells in exponential phase, so we need to consider the doubling time of the bacteria used.
4. After incubation, transfer the cells to a centrifuge tube and centrifuge at 4500 rpm for 10 minutes. Discard supernatant. Add fresh MH-II broth to the centrifuge tube and adjust the optical density of the cells to 0.5.
5. Transfer an aliquot (5 mL) of cells with adjusted optical density to a new centrifuge tube (50 mL). Add 3.75 μL of compound 053 stock solution (1000 μg/mL) to this tube. Cap the tubes and shake thoroughly to mix the compound. Then keep the centrifuge tube on the table for 15 minutes at room temperature. Note: It is preferable to have a larger surface area to facilitate agitation. The final concentration of 053 used in the experiment is 0.75 μg/mL. Do not incubate for more than 15 minutes.
6. After 15 minutes contact time, centrifuge the tubes for 10-15 minutes at 4500 rpm to obtain cell pellets and discard supernatant. Using 5 mL of phosphate- or potassium phosphate-buffered saline (PBS, pH7), wash the cell pellets thoroughly (PBS, pH7), repeating this operation twice. Note: Pipette several times with a 5 mL or 1 mL pipette during washing. Make sure there are no lumps during pipetting. The purpose of washing is to remove excess dye from the cell surface.
7. The color variation in the cells is visible to the naked eye on the cell pellet after the two washes.
a. Cells overexpressing the efflux pump (that is, resistant ones) will be white or off-white (default cell color).
b. Cells with normal or moderate efflux activity will be slightly pink in color.
c. Cells without efflux pumps or with dysfunctional efflux pumps will be colored dark pink.
d. Sometimes, cell pellets are orange in color, indicating moderate efflux activity, mainly due to the inherent yellow color of the cells.

### Results

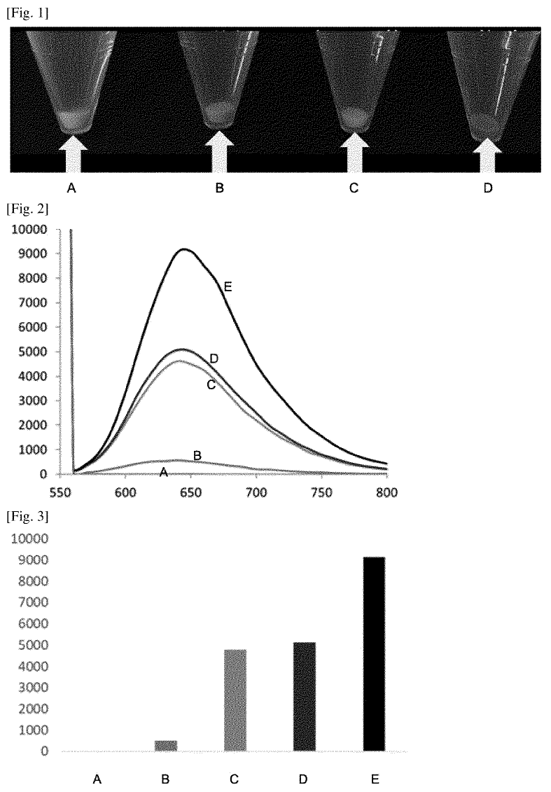

FIGS. 1 to 3 show the color difference (1A) and spectrofluorometric difference (1B) observed in different reference strains (strain A, CIP 76.25), a clinical strain (strain B, SA1199) and laboratory-built norA efflux pump mutants of strain B: NorA overexpressing (strain C, SA 1199B) and NorA deleted (strain D, SA K1758). Since strain C has an overexpressed efflux pump, 053 efflux is stronger, and the cell pellet appears white (053 is completely eliminated by efflux). However, as the strain D has no efflux pump, the cell pellet appears dark pink. Intermediate colors can be observed for reference strains (strain A) and clinical strains (strain B), suggesting mild to moderate activity. This is confirmed by fluorescence spectroscopy (FIGS. 2 and 3). [FIG. 2] shows the fluorescence emission spectrum of compound 053 in bacterial cells after excitation at 530 nm. [FIG. 3] shows the histograms obtained by plotting the fluorescence intensity values at 642 nm obtained after excitation at 530 nm.

The results obtained using colorimetry (FIGS. 1 to 3) clearly show that compound 053 makes it possible to visually distinguish 3 levels of strain coloration:
1. Dark pink coloration corresponding to strains lacking the NorA efflux pump (SAK 1758).
2. No coloration for strains overproducing the NorA pump (SA 1199B).
3. An intermediate pale pink coloration for strains producing basal pump levels (SA 1199 and CIP 76.25).

It is therefore possible to identify which isolates are overproducing the NorA efflux pump, and therefore have a very high potential for antibiotic resistance, and to differentiate them from isolates producing a basal level.

Spectrofluorimetric results ([FIG. 2]) show that compound 053 fluoresces when it accumulates in bacteria and that, like coloration, fluorescence depends on the level of NorA pump production.
1. The fluorescence of the overproducing strain (SA 1199B) is almost nil (FIGS. 2 and 3, green curve and histogram).
2. The NorA pump-free mutant (SAK 1758) has high fluorescence at 670 nm (FIGS. 2 and 3, black curve and histogram).
3. The two strains producing low levels of pumps (CIP 76.25, SA 1199) show intermediate levels of fluorescence (FIGS. 2 and 3, red and blue curves and histograms).

Unlike the colorimetric test, which requires no special reading equipment, fluorescence requires an appropriate spectrofluorometer. Fluorescence measurement, on the other hand, is quantitative and allows precise determination of the level of efflux activity of the isolates.

The inventors also carried out a fluorescence reading by flow cytometry in order to validate the results previously obtained by spectrofluorimetry. Advantageously, flow cytometry considers only the fluorescence within the cell and not surrounding it, thus enabling direct assessment of the concentration of compound 053 within the cell without the need for rinsing.

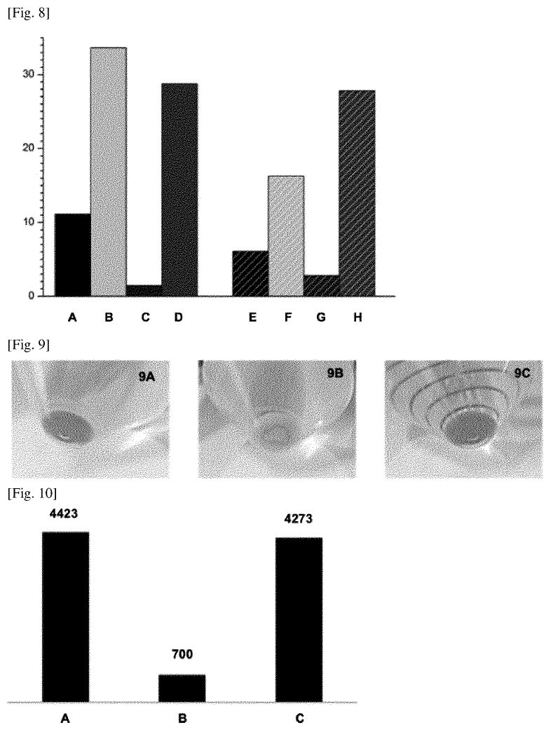

The results are consistent with those obtained previously. [FIG. 7] shows the fluorescence intensity measured by flow cytometry of a wild-type strain corresponding to CIP 76.25 (WT-A), a reference strain corresponding to SA (Ref strain—B), a NorA overproducer strain corresponding to SA1199B (NorA overproducer—C) and an norA-deleted strain corresponding to SA K1758 (NorA deleted—D). Cytometry results showed that compound 53 accumulated moderately in the wild-type and reference strains, accumulated very little in the NorA-overexpressing strain, and accumulated heavily in the norA-deleted strain. The inventors also tested the impact that a fixing agent such as 1% w/v paraformaldehyde (PFA) or 70% cold ethanol might have on cells treated with compound 053. [FIG. 8] shows the relative fluorescence intensity for each strain treated with compound 053 and with PFA (A-D) or ethanol (E-H). Whatever the binding agent used, the efflux differential between the different strains remains visible, proving the robustness of the method regardless of the binding environment.

### Example 2-2: Bacillus subtilis

### Materials and Methods

Bacterial strains: The inventors used
the reference strain Bacillus subtilis 168 (DSM 402) producing a basal level of BmrA,
Bacillus subtilis BmrA+++ overproducing the BmrA pump and
Bacillus subtilis BmrA Δ with the BmrA gene deleted.
(see Steinfels E et al. Biochemistry 43:7491-7502 and Krügel Het al. FEMS Microbiol Lett. 2010 December; 313(2): 155-63).

The protocols used are identical to those used in example 2-1.

### Results

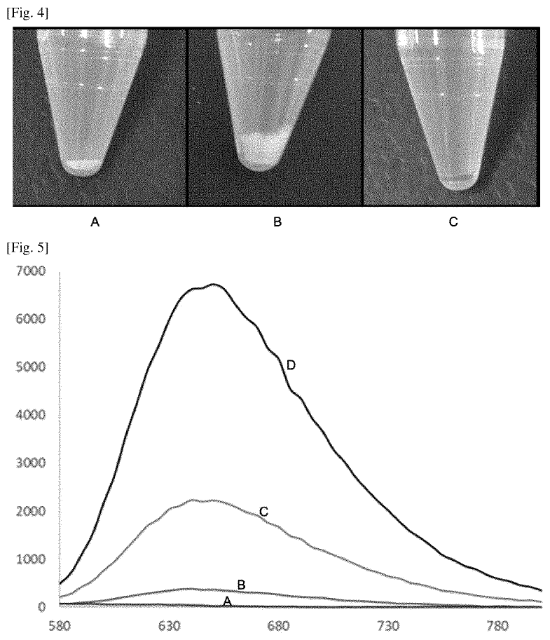

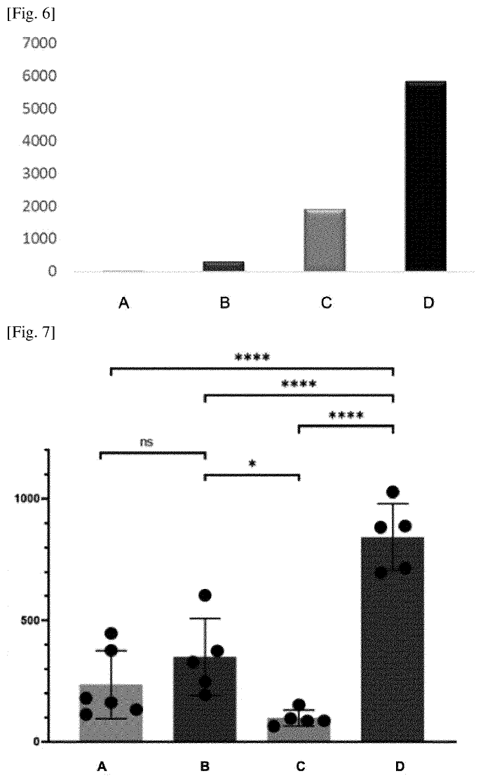

FIGS. 4 to 6 show the color difference ([FIG. 4]) and the spectrofluorometric difference (FIGS. 5 and 6) observed on the different Bacillus subtilis strains. Strain A is the wild-type strain of B. subtilis 168 (DSM 402), strain B, BmrA+++, is a mutant overexpressing BmrA, and strain C is a BmrA-deleted mutant (BmrAΔ). Since strain B shows overexpression of the BmrA efflux pump, compound 053 is completely effluxed out of the cell, and the cell pellet appears white. However, for strain C which has no efflux pump, the cell pellet appears dark pink. Strain A is a normal wild-type strain expressing normal efflux activity; compound 053 is therefore partially effluxed and the cell pellet appears pale pink, suggesting mild to moderate activity. Similar activity is confirmed and quantified using fluorescence spectroscopy (FIGS. 5 and 6).

As observed on [FIG. 4] in colorimetry, it is clear that compound 053 visually distinguishes the BmrA mutant at three coloration levels:
1. A dark pink coloration corresponding to strains lacking the BmrA efflux pump (BmrAΔ),
2. No coloration for strains overproducing the BmrA pump (BmrA+++)
3. Intermediate pale pink coloration for strains producing basal (normal, wild-type) levels of pump.

It is therefore possible to identify, among clinical isolates, those that overproduce the BmrA efflux pump, a medium-sized ABC (ATP-binding cassette) transporter involved in multidrug resistance.

The spectrofluorimetric results (FIGS. 5 and 6) are consistent and quantitatively validate the results. Compound 053 fluoresces when it accumulates in bacteria and, like coloration, fluorescence depends on the level of BmrA pump production.
1. The fluorescence of the overproducing strain (BmrA+++) is almost nil (FIGS. 5 and 6, curve and histogram)
2. The pump-free mutant (BmrAΔ) is very high, with a maximum at 642 nm (FIGS. 5 and 6, curve and histogram).
3. The wild-type strain (DSM 402) producing low levels of pumps shows intermediate levels of fluorescence (FIGS. 5 and 6, curve and histogram).

Unlike the colorimetric test, which requires no special reading equipment, fluorescence requires an appropriate spectrofluorometer. Fluorescence measurement, on the other hand, is quantitative and allows precise determination of the level of efflux activity of the isolates.

### Example 2-3: Streptococcus pneumoniae

The inventors have compared the staining induced by compound 053 on different strains of Streptococcus pneumoniae bacteria: a wild-type strain (“WT”), a strain overexpressing PatA-PatB, a Streptococcus pneumoniae ABC transporter for transporting molecules outside the cell involved in this pathogen's resistance to fluoroquinolone antibiotics (“OVP”), and a strain with PatA-PatB deleted (“DEL”).

### Materials and Methods

Bacterial strains: The inventors used
WT: the Streptococcus pneumoniae reference strain producing a basal level of PatA-PatB
OVP: The PatA-PatB overproducing strain
DEL: The strain with gene coding for PatA-PatB deleted

The protocols used are identical to those used in example 2-1.

### Results

FIGS. 9 and 10 show the color difference ([FIG. 9]) and spectrofluorometric difference ([FIG. 10]) observed on the different strains of Streptococcus pneumoniae.

Since the OVP strain shows overexpression of the PatA-PatB transporter, compound 053 is strongly effluxed out of the cell, and the cell pellet appears white. In contrast, for the WT strain and the DEL strain, which lacks the PatA-PatB transporter, the cell pellet appears dark pink. Similar activity is confirmed and quantified using fluorescence spectroscopy ([FIG. 10]).

Compound 053 can therefore be used to qualitatively and quantitatively distinguish a strain overexpressing an ABC PatA-PatB transporter from a strain not overexpressing this transporter or not expressing it at all.

### Example 2-4: Escherichia coli

### a) Compounds 392 and 29.

The inventors have tested the use of compound 392 and compound 29 on different E. coli strains with varying levels of expression of the AcrAB efflux pump.

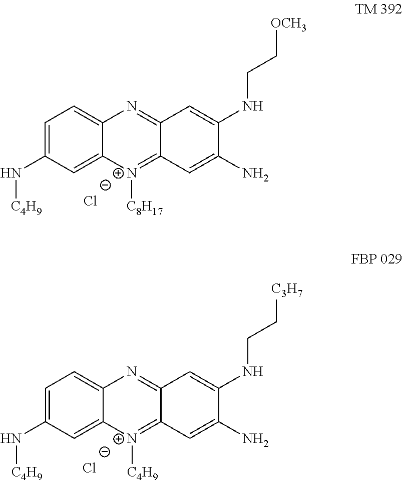

### Materials and Methods

The experimental protocol is the same as that described in the previous examples, except for the bacterial strains used. Briefly, compounds 29 and 392 (1 μg/mL) were added to 5 mL of a cell suspension of different E. coli strains (OD=1.5), mixed by tilting and left to stand on the table at room temperature for 15 min. The cells were then centrifuged at 3500 rpm for 10 min and rinsed with potassium phosphate buffer (PBP). The cell pellet was washed only once.

### Bacterial Strains:

| Strain | Description | Abbreviation |
|---|---|---|
| EC(ATCC11775) | Reference bacteria with | EC(ref) |
| | normal efflux activity | |
| AG100 | Wild-type bacteria (parent) | WT |
| AG100A | Bacteria with acrAB deletion | Δ acrAB |
| AG102 | Bacteria overexpressing acrAB | acrAB+++ |

### Results

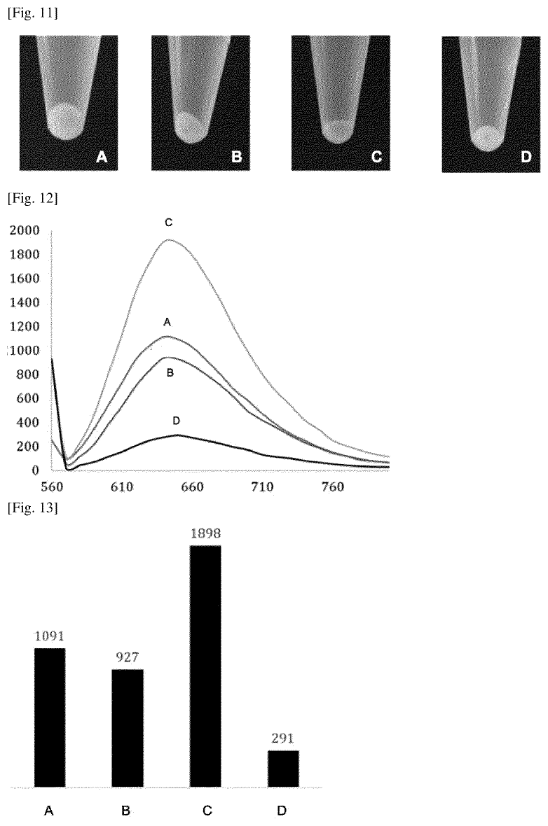

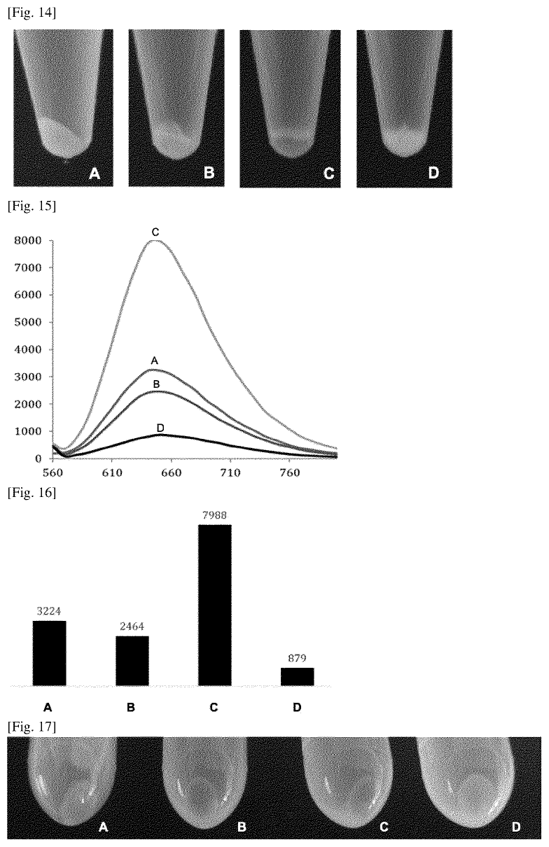

FIGS. 11 to 16 show the color difference (FIGS. 11 and 14) and the spectrofluorometric difference (FIGS. 12, 13, 15 and 16) observed on the different Escherichia coli strains. Strain EC (ref) is the reference strain (ATCC11775) showing normal efflux, strain AG100 is the wild-type strain of E. coli (Wild) also showing normal efflux, strain AG102 is an AcrAB overexpressing mutant (acrAB+++) showing strong efflux, and strain AG100A (Δ acrAB) is an acrAB-deleted mutant showing weak efflux.

As can be seen in FIGS. 11 and 14, bacteria with normal efflux (EC and AG100) are marked with a very pale pink, while the strain with the gene encoding the AcrAB pump deleted (AG100A) shows a dark pink stain. In contrast, the strain with AcrAB overexpression (AG102) is unstained. The same difference in efflux activity between the different strains is confirmed and even more noteworthy by fluorescence spectroscopy (FIGS. 12, 13, 15 and 16).

### b) Compound 053

The inventors also tested compound 053 (1 μg/mL) on the same Escherichia coli strains with varying levels of AcrAB efflux pump expression. The protocol was slightly modified to introduce a pre-treatment step with Carbonyl Cyanide m-Chlorophenylhydrazone (CCCP). CCCP is a chemical inhibitor of oxidative phosphorylation. It interferes with the transmembrane electrochemical gradient and proton-motive force, and consequently reduces ATP production. The effect of CCCP is to reduce the efficiency of efflux pumps, making it easier to perceive differences in staining between different strains.

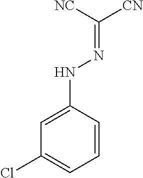 [SMILES: N#CC(C#N)=NNc1cccc(Cl)c1]

### Chemical Structure of CCCP

### Materials and Methods

The experimental protocol is the same as that described in the examples, with the difference that cells are treated for 1 hr with CCCP (5 μg/mL) in phosphate buffer (PPB). After CCP treatment, the whole experiment is run in PPB instead of MH-II broth from step 4 onwards.

### Results

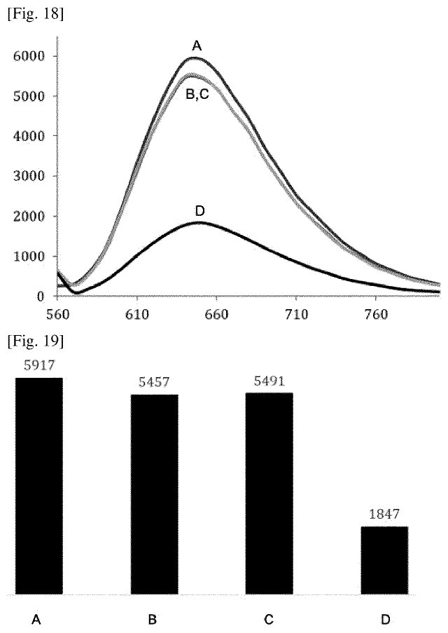

As can be seen in [FIG. 17], it is visually possible to distinguish strains with normal efflux (EC, AG100) or no efflux (AG100A) from the strain overexpressing AcrAB (AG102). Fluorescence spectroscopy (FIGS. 18 and 19) confirms this observation.

1. A compound of the following formula I: 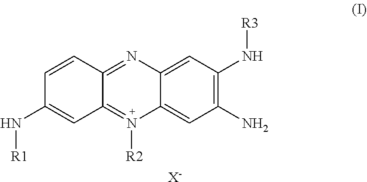
where X⁻ is a halogen atom, or an acceptable counter ion, in particular Cl,
where R1 and R2 are
either together —CH₃, or —C₄H₉
or a C₁-C₄ alkyl, such that if R1 is a Cₙ₊ᵢ alkyl, R2 is a Cₙ alkyl, and vice versa, n being an integer ranging from 1 to 3, i being an integer ranging from 1 to 3 and where n+i ranges from 1 to 4;
or R1 is —C₄H₇ and R2 is C₈H₁₇,
and
where R3 is a —CH₃, or a C₂-C₈ alkyl, said C₂-C₈ alkyl being optionally substituted with one or more heteroatoms;
or a salt or solvate of this compound of formula I, or a protonated form thereof.

2. The compound according to claim 1:
where R1 and R2 are
either together —CH₃,
or a C₁-C₄ alkyl, such that if R1 is a Cₙ₊ᵢ alkyl, R2 is a Cₙ alkyl, and vice versa, n being an integer ranging from 1 to 3, i being an integer ranging from 1 to 3 and where n+i ranges from 1 to 4;
and
where R3 is a —CH₃, or a C₂-C₈ alkyl, said C₂-C₈ alkyl being optionally substituted with one or more heteroatoms;
or a salt or solvate of this compound of formula I, or a protonated form thereof.

3. The compound according to claim 1 or claim 2, wherein R1 and R2 are each a C2-C4 alkyl, such that:
if R1 is a Cₙ₊ᵢ alkyl, R2 is a Cₙ alkyl, or
if R2 is a Cₙ₊ᵢ alkyl, R1 is a Cₙ alkyl,
n ranging from 1 to 3, i being an integer ranging from 1 to 3 and where n+i ranges from 1 to 4.

4. The compound according to any one of claims 1 to 3, where R3 is C2-C4 alkoxy, dimethylaminoethyl or methylaminexethyl.

5. The compound according to any one of claims 1 to 4, said compound being selected from the group of compounds of formula I, wherein R1, R2 and R3 are together are as indicated in each row of the following table:
TABLE 1

| compound | R1 | R2 | R3 |
|---|---|---|---|
| | methyl | methyl | propyl |
| | methyl | ethyl | propyl |
| | ethyl | methyl | propyl |
| | ethyl | propyl | propyl |
| | propyl | ethyl | propyl |
| | propyl | butyl | propyl |
| | butyl | propyl | propyl |
| | methyl | methyl | butyl |
| | methyl | ethyl | butyl |
| | ethyl | methyl | butyl |
| | ethyl | propyl | butyl |
| | propyl | ethyl | butyl |
| | propyl | butyl | butyl |
| #47 | butyl | propyl | butyl |
| | methyl | methyl | methoxyethyl |
| | methyl | ethyl | methoxyethyl |
| | ethyl | methyl | methoxyethyl |
| | ethyl | propyl | methoxyethyl |
| | propyl | ethyl | methoxyethyl |
| | propyl | butyl | methoxyethyl |
| #53 | butyl | propyl | methoxyethyl |
| #52 | butyl | propyl | octyl |
| #392 | butyl | octyl | methoxyethyl |
| #29 | butyl | butyl | propyl |

6. The compound according to any one of claims 1 to 5, said compound being selected from compounds of the following formulae 47, 52, 53, 392 and 29:

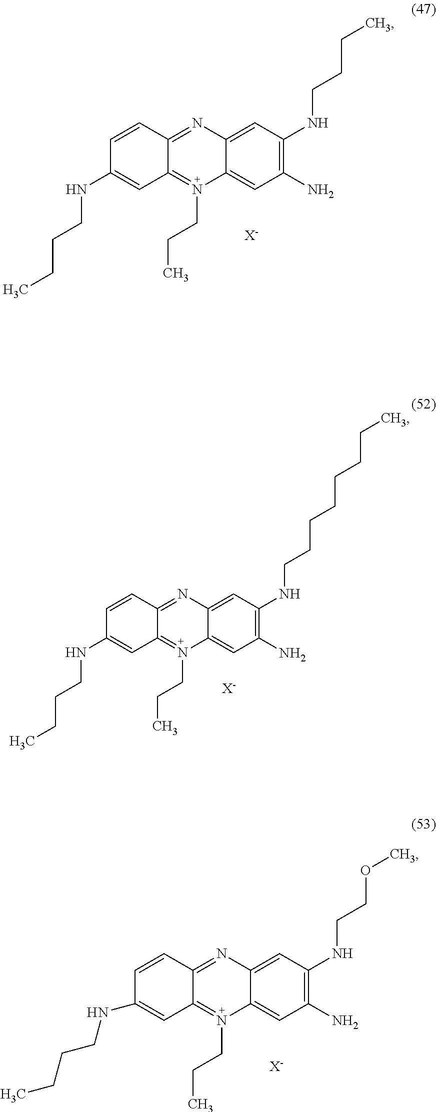

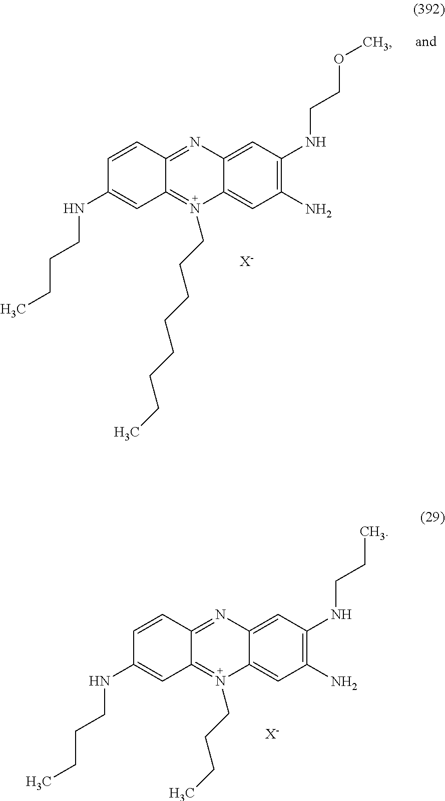

7. A use of a compound according to any one of claims 1 to 6, to evaluate or measure the efflux of said compound by a bacterium.

8. The use according to claim 7, to evaluate or measure efflux by Gram-positive bacteria.

9. The use according to claim 7, to evaluate or measure efflux by Gram-negative bacteria.

10. A bacterial efflux detection or measurement kit, comprising
a compound according to any one of claims 1 to 6, and
at least one control bacterium.

11. The detection kit according to claim 10, additionally comprising a protonophore, notably chosen from 2,4-dinitrophenol, Carbonyl cyanide-p-trifluoromethoxyphenylhydrazone (FCCP), carbonyl cyanide m-chlorophenyl hydrazone (CCCP), C4R1, ellipticine, 10-[2-(3-hydroxy-6-oxo-xanthen-9-yl)benzoyl]oxydecyl-triphenyl-phosphonium bromide and 2-(2-Hydroxyaryl)hexylphosphonium bromide.

12. The use of a compound according to any one of claims 1 to 6, to assess the resistance of a bacterium to a bacteriostatic or bactericidal compound.

13. A method for assessing resistance due to an efflux mechanism of a bacterium to a bacteriostatic or bactericidal compound, comprising:
cultivating bacteria in a suitable culture medium, to obtain a bacterial culture,
bringing the bacterial culture into contact with a compound as defined in any one of claims 1 to 6, to obtain a treated bacterial culture,
washing the treated bacterial culture, and precipitating the bacteria contained in the treated bacterial culture to obtain a bacterial pellet,
observing the staining of the bacterial pellet, such that
if the bacterial pellet has the color of a bacterial pellet from the untreated bacterial culture, said bacteria are resistant to said bacteriostatic or bactericidal compound, by efflux of said bacteriostatic or bactericidal compound, and
if the bacterial pellet has a color different from the color of a bacterial pellet from the untreated bacterial culture, said bacteria are not resistant to said bacteriostatic or bactericidal compound, by efflux of said bacteriostatic or bactericidal compound.

14. The method according to claim 13, said method further comprising a step of incubating the bacterial culture with a protonophore.
